(19) United States
(12) Patent Application Publication
(10) Pub. No.: US 2022/0301681 A1
(43) Pub. Date: Sep. 22, 2022

(54) System and Method for Infusion of Drugs

(71) Applicant: University of Cape Town, Cape Town (ZA)

(72) Inventor: Ettienne Coetzee, Cape Town (ZA)

(21) Appl. No.: 17/634,482

(22) PCT Filed: Aug. 13, 2020
(86) PCT No.: PCT/IB2020/057614

§ 371 Date: Feb. 10, 2022

(30) Foreign Application Priority Data
Aug. 14, 2019 (GB) 1911611.0

(51) Int. Cl. G16H 20/17; A61M 5/142; A61M 5/145; G16H 40/63

(52) U.S. Cl. CPC G16H 20/17; A61M 5/1424; A61M 5/1452; G16H 40/63; A61M 2005/14208

(57) ABSTRACT

The invention provides a user-guidance device for informing a required change to a pump volume displacement parameter of a manual drug infusion pump assembly, for use with manually-controlled infusion of a drug into a patient. The user-guidance device comprises a measurement component arranged to measure the pump volume displacement parameter. The device further comprises a computing device arranged to calculate a measured rate of infusion of the drug based on the measured pump volume displacement parameter; and to calculate with reference to said measured rate of infusion and to at least one model selected from the group consisting of pharmacokinetic and pharmacodynamic models, a model-simulated drug concentration in the patient. The computing device may be arranged to derive user-guidance information based on the model-simulated drug concentration. The invention also provides a drug infusion system which includes the disclosed user-guidance device in combination with a manual drug infusion pump assembly.

### CROSS-REFERENCE TO RELATED APPLICATION

This application claims priority from United Kingdom patent application number 1911611.0 filed on 14 Aug. 2019, which is incorporated by reference herein.

### FIELD OF THE INVENTION

This invention relates to the infusion of drugs. It relates in particular, although not exclusively, to the infusion of anaesthetic agents.

### BACKGROUND TO THE INVENTION

Drug infusion may be performed manually or automatically. In manual techniques a clinician makes each change to the rate of infusion. In automatic techniques such as target-controlled infusion (TCI), the clinician sets a target blood or effect-site concentration and a computerised, autonomous infusion device makes necessary changes to the infusion rate in order to achieve the target concentration in the applicable body compartment or tissue.

In the field of anaesthesia, continuous infusion of intravenous anaesthetic agents is commonly used to induce and maintain sedation and general anaesthesia. Intravenous anaesthetic agents reach clinical effect after an appropriate drug concentration is established in the bloodstream or at an effect-site such as the patient's brain.

Drugs have pharmacokinetic (PK) and pharmacodynamic (PD) properties which affect drug concentration levels, dosing, benefit, and adverse effects. Since rapid and ongoing measurement of drug concentration is not routinely feasible, PK principles can be applied to calculate how much of an anaesthetic agent has accumulated in tissues and to predict concentrations and required infusion rates. This information can be applied to adjust the infusion rate to maintain a stable concentration in blood plasma or the tissue of interest. Target-controlled infusion devices or pumps (“TCI devices”) are available which can control infusion with reference to mathematical calculations based on PK mathematical models. However, TCI devices are regarded as high-risk medical devices and are required to pass stringent and lengthy regulatory compliance protocols to ensure the safety and efficacy of their autonomous functioning. These regulatory hurdles increase the costs of such devices and cause delays so that, by the time such devices reach the market, the PK models upon which they are based may be out of date.

Instead of using TCI devices; clinicians may perform manually controlled infusions with the aid of a syringe or a non-PK syringe pump set to dispense the drug of interest at a zero-order (constant) infusion flow rate, PK software that may be running on a separate computer or smart device may be consulted to provide advice or guidance during the infusion procedure.

EP0164904A filed on 10 May 1985 and entitled “Open-loop control of drug infusion” discloses a method of determining a generalised infusion rate profile for the delivery of drugs. The method comprises the steps of (a) infusing a drug at arbitrary but known rates into a group of patients for each of whom the Lean Body Mass has been determined; (b) determining the plasma arterial concentration of the drug in each patient at a number of specific time intervals throughout each infusion period; (c) for each patient, estimating the rates of loss of the drug from the circulation of the patient at a number of specific time instants by dividing the known infusion rates per Lean Body Mass of these instants by the plasma arterial concentrations of the drug at each of these instants; (d) calculating the average of the estimated rates of loss of drug from the circulation per Lean Body Mass unit at each specific time interval for the group of patients; (e) interpolating the successive average points between the specific time intervals to produce an infusion profile; (f) infusing said drug in accordance with said infusion profile determined from said interpolations into a group of patients for each of whom the Lean Body Mass has been determined, said infusion rate being scaled according to said Lean Body Mass of each patient, and (g) repeating steps (b) to (f) until a desired steady plasma arterial content of the drug is substantially maintained throughout the infusion period.

TW201437835A filed on 20 Mar. 2013 and entitled “Fast calculation method for target-controlled manual administration and apparatus thereof” discloses a method of calculating an optimal instantaneous injection dosage and the best continuous infusion rate. The aim of the invention was to find an optimal instantaneous dosage to bring the concentration of a drug at its site of action to a target value; and to find the best continuous infusion rate for maintaining the concentration at the target value thereafter. The objective was to reduce the number of manual control adjustments required to be made to the infusion rate. The disclosed method involves the steps of providing a program platform in an electronic device and loading it with a fast calculation program configured by the user based on a target value of the concentration at the action site; and calculating the optimal instantaneous injection dosage and the best continuous infusion rate according to a pharmacokinetic model. The method uses an algorithm based on real root separation of polynomial exponential functions to calculate all possible concentration trends at the site of action generated by the instantaneous injection dosage and continuous infusion rate and determines the optimal one to prompt the user to perform manual administration. The method uses a binary search method to reduce the number of calculations for concentration trends at the site of action.

The above methods—and manually controlled infusion approaches in general—have certain drawbacks. They are inherently difficult to perform and cannot provide the clinician with feedback regarding the progress of the infusion; errors are not measured and accordingly cannot be taken into account. Furthermore, to maintain accuracy the clinician must repeatedly enter administered drug dosages for processing by the PK software.

There is scope to address the aforementioned drawbacks and disadvantages. A need exists for infusion systems, devices and methods which can allow clinicians and patients to benefit from the latest PK and PD models as they are developed, and to restrict costs by limiting the need for lengthy and costly regulatory approval protocols. A further need exists for systems, devices and methods to support manually controlled infusion techniques and to mitigate the risk of possible errors associated therewith.

There is also a need for new methods of compiling information and parameters for use in pharmacokinetic and pharmacodynamic studies, and for methods of constructing, validating and assessing the accuracy of pharmacokinetic and pharmacodynamic models. A further need exists for systems and methods capable of monitoring and validating the operational accuracy of infusion pumps, e.g. target-controlled infusion (TCI) devices.

The preceding discussion of the background to the invention is intended only to facilitate an understanding of the present invention. It should be appreciated that the discussion is not an acknowledgment or admission that any of the material referred to was part of the common general knowledge in the art as at the priority date of the application.

### SUMMARY OF THE INVENTION

According to a first aspect of the invention there is provided a user-guidance device for informing a required change to a pump volume displacement parameter of a manual drug infusion pump assembly, for use with manually-controlled infusion of at least one drug into a patient, said user-guidance device comprising:
at least one measurement component arranged to measure the pump volume displacement parameter; and
a computing device arranged to:
   calculate a measured rate of infusion of the drug into the patient based on the measured pump volume displacement parameter; and
   calculate with reference to said measured rate of infusion and to at least one model selected from the group consisting of pharmacokinetic and pharmacodynamic models, a model-simulated drug concentration in said patient.

The computing device may be arranged to derive user-guidance information based on said model-simulated drug concentration. It may be arranged to output the user-guidance information.

According to a further aspect of the invention there is provided a drug infusion system which includes a user-guidance device as disclosed herein and a manual drug infusion pump assembly.

Throughout the specification and claims, the term “manual drug infusion pump assembly” will be understood to mean a manually controllable drug infusion pump assembly arranged to dispense a drug at manually-variable flow rates, but lacking functionality for automatically varying flow rates in reference to models selected from the group consisting of pharmacokinetic (PK) and pharmacodynamic (PD) models. Without limiting the generality thereof, the term will be understood to encompass an assembly comprising a syringe, and an assembly comprising a combination of at least one syringe with a non-pharmacokinetic, zero-order infusion pump. The syringe may comprise a standard anaesthesia syringe. The zero-order infusion pump may comprise a manually controllable syringe pump.

The pump assembly may accordingly be selected from the group consisting of syringes and non-pharmacokinetic, zero-order, manually controllable syringe pumps in combination with syringes.

The user-guidance device may be referred to in the alternative as a target-guided infusion (TGI) device.

The computing device may be arranged to calculate, with reference to the pump volume displacement parameter and the model, a rate of infusion required for achieving in said patient a drug concentration within a target concentration range. The computing device may be arranged to compare the measured rate of infusion against the required infusion rate, thereby to establish a deviation of the measured infusion rate from the required infusion rate; and generate deviation notification data if said deviation breaches a definable threshold.

The computing device may be arranged to generate deviation notification data if the model-simulated drug concentration breaches a definable threshold concentration.

The system may include at least one alerting component configured to trigger a user-discernible alert signal responsive to the generation of the deviation notification data.

The drug may be selected from the group consisting of anaesthetic, hypnotic, analgesic and neuromuscular blocking agents. The drug may be selected from intravenously administered drugs.

The computing device may include a processing component and a memory configured to provide computer program instructions as software units to the processor to execute the calculations and comparisons referred to above.

The user-guidance or TGI device may include a user interface (UI) component comprising an input component and an output component. The output component may include at least one alerting component configured to trigger a user-discernible alert or alarm signal responsive to the generation of the deviation notification data. Without limitation thereto, the output component may be arranged to generate the alert or alarm signal by means selected from the group consisting of visual, audio, haptic and print means. The alerting component may, in use, serve to alert a user of the system that manual setting or operation of the pump assembly is required to adjust the rate of infusion.

The output component may include a display component, which may be arranged to display results of the calculations and comparisons performed by the computing device.

The target concentration range and the model-simulated drug concentration may comprise a concentration at a site selected from the patient's body compartments and tissues, including, without limitation thereto, the patient's blood plasma, spinal cord, central nervous system and brain. The concentration may be a plasma-site concentration. The concentration may be an effect-site concentration.

Without limitation thereto, the input component of the user interface component may be arranged to receive information representative of input parameters selected from the group consisting of:
at least one pump volume displacement parameter (e.g. linear travel of a syringe plunger);
at least one target drug concentration;
type and starting concentration of at least one drug to be administered from the manual pump assembly;
patient characteristics;
characteristics of the manual pump assembly (e.g. syringe dimensions, volume and/or size); and
effect level information for at least one type of drug effect to be achieved in the patient, which information may include at least one effect level value selectable from a predetermined effect scale.

The memory component of the computing device may be arranged to store the information representative of the input parameters. The computing device may be arranged to perform the calculations with reference to at least some of the input parameters.

The computing device may be arranged to update the model-simulated drug concentration and the model-simulated, required infusion rate responsive to variations in the measured rate of infusion. The computing device may be arranged to perform the updating by execution of a feedback loop. The output component may be arranged to output, in addition to the results of the calculations made initially, results of updates made to such calculations.

The computing device may be arranged to calculate time-to-recovery or emergence of the patient. The computing device may be arranged to calculate at least one clinical effect of the infused drug or drugs.

The user-guidance or TGI device may be adapted for retro-fitting to the pump assembly. The TGI device may accordingly serve as a so-called after-market accessory for use with a zero-order infusion pump assembly such as a syringe or a combination of a syringe and a manually controllable syringe pump. Advantageously, at least a portion of the TGI device may be configured for tool-free engagement with, and disengagement from, the pump assembly. The TGI device may accordingly comprise engagement formations suitable for detachably securing at least a portion of the TGI device to the pump assembly.

The user-guidance or TGI device may include a body comprising a pair of hingedly or pivotably connected body portions for closing over the pump assembly and locking it to the TGI device.

The user-guidance or TGI device may be of a unitary construction. To this end, the computing device and the measurement component may be at least partially integrated with each other. The computing device and the measurement component may be fully integrated with each other.

The computing device and the measurement component may be housed together with each other. They may be attached to each other.

The user-guidance or TGI device may instead have a spatially distributed arrangement, whereby the computing device and the measurement component are provided separately from one another. The computing device and the measurement component may be connectable to each other by means of suitable wired or wireless data communication means.

The definable thresholds of concentration and infusion rate may be characterised as definable error margins. The input parameters may include a parameter for the threshold or error margin which can be selected and set by a user of the system via the input component. Instead or in addition, the threshold or error margin may be provided stored on a computer-readable medium. The computer-readable medium may be a non-transitory computer-readable medium.

The threshold or error margin may be representative of a maximum clinically-acceptable deviation of the calculated, model-simulated concentration from the definable threshold concentration (i.e. from a target concentration at a plasma site and/or effect-site). The threshold or error margin may be representative of a maximum clinically-acceptable deviation of the measured rate of infusion from the required infusion rate (i.e. from a target infusion rate).

The computing device may be arranged to calculate at least one value selected from the group consisting of:
bolus doses (expressed in units of volume and/or mass) required to be infused to bring the model-simulated drug concentration to within the target concentration range and inside the error margin;
infusion pause durations required to be waited by the user after infusion of a first bolus, before initiating an infusion or administering a second bolus to maintain the model-simulated drug concentration within the target concentration range and inside the error margin; and
infusion rates required to maintain the model-simulated concentration within the target concentration range and inside the error margin.

The derivable user-guidance information based on the model-simulated drug concentration may be selected from the group consisting of: calculated required rates of infusion; calculated updates to required infusion rates; required bolus volumes; required infusion pause durations; deviation notification data; alert signals; calculated time to recovery; calculated time to emergence; calculated clinical effect; and calculated drug interactions.

It will be appreciated that, whilst the computing device is advantageously provided as a dedicated and typically stand-alone device, at least some of the components thereof may be provided by remotely-locatable computing apparatus. The remotely-locatable apparatus may be selected from the group consisting of smartphones, laptop computers, tablet computers, other mobile computing devices, servers, distributed computing arrangements, cluster computing arrangements, and cloud-based computing arrangements.

The measurement component may include displacement sensing means for measuring the volume displacement parameter of the drug infusion pump assembly, thereby to facilitate measurement of drug volumes dispensed from the pump assembly.

The volume displacement parameter may be a positional parameter. It may be a parameter representing linear displacement of a syringe plunger within a syringe barrel. In such a case, the displacement sensing means may comprise a linear displacement sensor for measuring a linear displacement of the syringe plunger within the syringe barrel. It will be appreciated, however, that numerous other types of displacement sensing means may be implemented in the measurement component, as further described elsewhere herein.

The linear displacement sensor may comprise a rectilinear potentiometer or rheostat.

The measurement component may further comprise means for automatically detecting the dimensions of a syringe, and for transmitting associated values to the input component of the computing device. During use of such embodiments, the input parameters characteristic of the manual drug infusion pump assembly may be at least partially provided by the measurement component.

The drug infusion system may comprise a plurality of pump assemblies for concurrently or simultaneously infusing a plurality of drugs. It may further comprise a plurality of measurement components, each being associated with one of the pump assemblies, so that respective pump volume displacement parameters associated with the respective pump assemblies may be measured. The measurement components may be arranged in data communication with the computing device. The computing device may be arranged to calculate data relating to interactions between the drugs, and with reference to said data and the model, to calculate model-simulated drug concentrations and a required change to a rate of infusion of at least one of the drugs, taking account of the drug interactions and the pump volume displacement parameters. The computing device may further be arranged to predict clinical end points based on said data.

The measurement components may be arranged in data communication with the computing device. The computing device may be arranged to calculate data relating to interactions between the drugs

In such embodiments of the system, either or both of the pharmacokinetic and pharmacodynamic models may include predictions with regard to at least one effect selected from the group consisting of additive, synergetic and synergistic effects of at least two drugs.

The user-guidance device may be referred to as a target-guided infusion (TGI) device. The user-guidance or TGI device may include a user interface component comprising an input component and output component. The output component may include at least one alerting component configured to trigger a user-discernible alert or alarm signal responsive to generation of the deviation notification data. Without limitation thereto, the output component may be arranged to generate the alert or alarm signal by means selected from the group consisting of visual, audio, haptic and print means. The alerting component may, in use, serve to alert a user of the system that manual setting or operation of the infusion pump assembly is required to adjust the rate of infusion.

Details of the computing device, its components and its calculations, the user interface component, the pump assembly, the drug and sites of concentration may be as hereinbefore described.

According to a further aspect of the invention there is provided a computer implemented method of infusing at least one drug into a patient, which includes:
utilising at least one manual drug infusion pump assembly having a pump volume displacement parameter;
infusing the drug into the patient using the manual drug infusion pump assembly;
providing at least one measurement component arranged to measure the pump volume displacement parameter of the pump assembly;
measuring said pump volume displacement parameter using the measurement component; and
providing at least one computing device and operating it to:
   calculate a measured rate of infusion of the drug into the patient based on the measured pump volume displacement parameter; and
   calculate with reference to said measured rate of infusion and to at least one model selected from the group consisting of pharmacokinetic and pharmacodynamic models, a model-simulated drug concentration in said patient.

The method may further include deriving user-guidance information based on said model-simulated drug concentration. It may further include outputting the user-guidance information.

The measurement component and the computing device may be connectable with each other to provide a user-guidance device, also referred to herein as a target-guided infusion (TGI) device.

The method may include generating deviation notification data if the model-simulated drug concentration, calculated with reference to the measured rate of infusion and the model, breaches a definable threshold concentration.

The method may include calculating, with reference to the pump volume displacement parameter and the model, a model-simulated infusion rate required for achieving, in said patient, a drug concentration within a target concentration range. The method may include administering at least one drug at or near the required infusion rate determined for that drug.

The method may include comparing the measured rate of infusion against the required infusion rate, thereby to establish a deviation of the measured infusion rate from the required infusion rate; and generating deviation notification data if said deviation breaches a definable threshold.

The method may include generating a user-discernible alert or alarm signal responsive to generation of the deviation notification data. Means selected from the group consisting of visual, audio, haptic and print means may be provided and the method may include generating the alert or alarm signal by such means.

The method may include manually regulating the infusion of the drug into the patient responsive to either or both of said deviation notification data and said alert signal.

The method may include engaging at least a portion of the user-guidance or TGI device with the pump assembly. The TGI device may be engaged with the pump assembly in a tool-free manner.

The method may include infusing a plurality of drugs from a plurality of pump assemblies and measuring volumes of the drugs dispensed from the respective pump assemblies. It may include providing at least one computing device arranged to calculate data relating to interactions between said drugs as well as model-simulated concentrations and required infusion rates which take account of said interactions and the measured volumes of drugs dispensed. The computing device may further be arranged to predict clinical end points based on said data.

The method may include arranging a plurality of measurement components, each associated with one of the pump assemblies, in data communication with the computing device so that volume displacement parameters pertaining to the pump assemblies may be measured and used to inform the data calculations relating to the drug interactions.

In such modes for performance of the method, either or both of the pharmacokinetic and pharmacodynamic models may include predictions with regard to at least one effect selected from the group consisting of additive, synergetic and synergistic effects of at least two drugs.

Details of the computing device, its components and its calculations, the user interface component, the pump assembly, the drug or drugs and sites of concentration may be as hereinbefore described.

The invention extends to a computer implemented method for informing a required change to a pump volume displacement parameter of a manual drug infusion pump assembly, the method including:
monitoring at least one manual drug infusion pump assembly having a pump volume displacement parameter;
measuring said pump volume displacement parameter;
providing a computing device and operating it to:
   calculate a measured rate of infusion of a drug into a patient based on the measured pump volume displacement parameter; and
   calculate with reference to said measured rate of infusion and to at least one model selected from pharmacokinetic and pharmacodynamic models, a model-simulated drug concentration in said patient.

The method may further include deriving user-guidance information based on said model-simulated drug concentration. It may include outputting the user-guidance information. It may include outputting the user-guidance information for control of manual drug infusion. The user-guidance information may be as hereinbefore described.

The method may include calculating, with reference to the measured rate of infusion and the model, a rate of infusion required for achieving, in said patient, a drug concentration within a target concentration range; comparing the measured rate of infusion against the required infusion rate, thereby to establish a deviation of the measured infusion rate from the required infusion rate; and generating deviation notification data if said deviation breaches a definable threshold.

The method may further include generating deviation notification data if the model-simulated drug concentration breaches a definable threshold concentration.
Accordingly, the method may include generating deviation notification data if either or both of the following conditions are met:
   (a) the model-simulated drug concentration breaches a definable threshold concentration;
   (b) a deviation of the measured rate of infusion from a required infusion rate occurs, said required infusion rate being a rate of infusion required for achieving, in said patient, a drug concentration within a target concentration range, and said deviation of the measured rate of infusion therefrom being established by performance of the following steps:
      calculating, with reference to the pump volume displacement parameter and the model, the required infusion rate; and
      comparing the measured rate of infusion against the required infusion rate, thereby to establish the deviation of the measured rate of infusion from the required infusion rate.

The method may include monitoring infusion of a plurality of drugs from a plurality of pump assemblies; measuring respective volumes of the drugs dispensed from the respective pump assemblies; operating the computing device to calculate information relating to interactions between said drugs, based on the measured volumes dispensed; and outputting said data as part of the user-guidance information.

In such modes for performance of the method, either or both of the pharmacokinetic and pharmacodynamic models may include predictions with regard to at least one effect selected from the group consisting of additive, synergetic and synergistic effects of at least two drugs.

The invention extends to a method of operating the disclosed user-guidance device (TGI device) to compile statistical information for a study selected from the group consisting of pharmacokinetic and pharmacodynamic studies; said method including steps of
monitoring infusion of a drug into a cohort of patients conducted by a manual drug infusion pump assembly, said pump assembly having a pump volume displacement parameter;
for each patient within said cohort of patients:
   operating the measurement component of the user-guidance device to measure the pump volume displacement parameter of the pump assembly;
   operating the computing device of the user-guidance device to calculate a measured rate of infusion of the drug into the patient based on the measured pump volume displacement parameter; and
   recording data pertaining to the measured rate of infusion;

thereby to provide statistical information relating to measured rates of infusion pertinent to the patients in the cohort.

The method may further include recording a time of measurement of the measured pump volume displacement parameter. The method may include recording a time of calculation of the measured rate of infusion. Thus, the method may include measuring and recording a time of infusion of the drug into each patient within the cohort. The method may include logging said times as part of the statistical information. Thus, the statistical information that is provided may additionally include times of infusion pertinent to the patients in the cohort.

The method may include outputting the statistical information.

Thus, the user-guidance or TGI device may be operable to record and keep accurate logging information in terms of drug administration and times of administration which may then be used to calculate pharmacokinetic parameters. Use of the TGI device may therefore simplify record keeping during the development of pharmacokinetic studies. When used in this fashion, the TGI device itself may not necessarily be required or used to calculate drug concentrations within patients.

The invention extends to a method of operating the disclosed user-guidance device to construct a model selected from the group consisting of pharmacokinetic and pharmacodynamic models; said method including steps of
monitoring infusion of a drug into a cohort of patients conducted by a manual drug infusion pump assembly, said pump assembly having a pump volume displacement parameter;
for each patient within said cohort of patients:
   operating the measurement component of the user-guidance device to measure the pump volume displacement parameter of the pump assembly;
   operating the computing device of the user-guidance device to calculate a measured rate of infusion of the drug into the patient based on the measured pump volume displacement parameter;
   recording data pertaining to the measured rate of infusion; and
   measuring and recording a concentration of the drug in the patient;
thereby to provide statistical information relating to measured rates of infusion and drug concentrations pertinent to the patients in the cohort.

The method may further include recording a time of measurement of the measured pump volume displacement parameter. The method may include recording a time of calculation of the measured rate of infusion. Thus, the method may include measuring and recording a time of infusion of the drug into each patient within the cohort. The method may include logging said times as part of the statistical information. Thus, the statistical information that is provided may additionally include times of infusion pertinent to the patients in the cohort.

The method may include outputting the statistical information.

The method may include mathematically manipulating the statistical information using a pre-defined algorithm adapted to cross-reference parameters selected from the group consisting of the measured rates of infusion, the times of infusion and the drug concentrations for the patients in the cohort, thereby to construct the model.

The invention extends further to a method of operating the disclosed user-guidance device for assessing accuracy of first and second models selected from the group consisting of pharmacokinetic and pharmacodynamic models; said method including steps of
monitoring infusion of a drug into a patient conducted by a manual drug infusion pump assembly operable to achieve a target drug concentration in the patient calculated by interrogation of the first model, said pump assembly having a pump volume displacement parameter;
operating the measurement component of the user-guidance device to measure the pump volume displacement parameter of the pump assembly;
operating the computing device of the user-guidance device to
   calculate a measured rate of infusion of the drug into the patient based on the measured pump volume displacement parameter; and
   calculate with reference to said measured rate of infusion and to the second model, a model-simulated drug concentration in said patient;
measuring and recording a concentration of the drug in the patient;
comparing the measured concentration of the drug in said patient with the target drug concentration and the model-simulated drug concentration; thereby to provide comparative information relating to the first and second models; and
outputting the comparative information to support assessment of the relative accuracy of the first and second models in relation to the measured concentration.

The invention extends further to a method of operating the disclosed user-guidance device for validation of the operational accuracy of an automated target-controlled infusion pump configured to drive a manual drug infusion pump assembly for infusing a drug into a patient to achieve a target drug concentration; said method including steps of
monitoring the infusion of the drug into the patient by operating the measurement component of the user-guidance device to measure a pump volume displacement parameter of the pump assembly;
operating the computing device of the user-guidance device to
   calculate a measured rate of infusion of the drug into the patient based on the measured pump volume displacement parameter; and
   calculate with reference to said measured rate of infusion and to at least one model selected from the group consisting of pharmacokinetic and pharmacodynamic models, a model-simulated drug concentration in said patient;
comparing the target drug concentration against the model-simulated drug concentration calculated by the user-guidance device, thereby to determine a deviation of the target concentration from the model-simulated concentration and provide a metric of the operational accuracy of said target-controlled infusion pump; and
outputting said metric to support the validation of the operational accuracy of said target-controlled infusion pump.

The invention extends to a computer program product comprising computer-readable program code with instructions arranged to cause the described computing device to execute the computer operable steps of at least one of the methods described above.

The computer program product may comprise a purpose-created mobile software application installable on a mobile device.

The invention extends to a computer-readable medium having stored thereon the computer program product described above. The computer-readable medium may comprise a non-transitory computer-readable medium. The computer-readable program code may be executable by the processor or processors of the computing device.

Embodiments and modes of performance of the invention will now be described, by way of example only, with reference to the accompanying drawings in which like reference numerals have been used to designate similar parts or features correspondingly throughout, unless otherwise stated.

### BRIEF DESCRIPTION OF THE DRAWINGS

In the drawings:

FIG. 1 illustrates, schematically, a handheld variant of a drug infusion system comprising a target-guided infusion (TGI) device and a manually-operable syringe;

FIG. 2 illustrates, schematically, an alternative variant of a drug infusion system comprising a TGI device mounted on an infusion pump assembly, the pump assembly consisting of a syringe and a manually controllable syringe pump capable of driving the syringe;

FIG. 3 illustrates, schematically, a first, handheld embodiment of a user-guidance or TGI device which forms part of the system variant shown in FIG. 1, the device being shown in a closed condition;

FIG. 4 illustrates, schematically, the handheld TGI device of FIG. 3, the device being shown in an open condition;

FIG. 5 illustrates, schematically, a right side view of the handheld TGI device of FIGS. 3 and 4, shown in closed condition;

FIG. 6 illustrates, schematically, a plan of the handheld TGI device of FIGS. 3 and 4, shown in closed condition;

FIG. 7 illustrates, schematically, a rear view of the handheld TGI device of FIGS. 3 and 4, shown in closed condition;

FIG. 8 illustrates, schematically, a right side view of the variant of the drug infusion system shown in FIG. 2, including a pump-attachable variant of a TGI device;

FIG. 9 illustrates, schematically, a plan of the pump-attachable variant of the TGI device shown in FIG. 8;

FIG. 10 illustrates, schematically, a rear view of the pump-attachable TGI device shown in FIGS. 8 and 9;

FIG. 11 is a simplified system block diagram which illustrates, schematically, a high-level overview of the described drug infusion system and components thereof;

FIG. 12 is a flow diagram which illustrates, schematically, a method for infusing a drug into a patient using the described drug infusion system;

FIG. 13 is a flow diagram which illustrates, schematically, a subprocess of the flow diagram of FIG. 11, for performing an initial setup of a TGI device;

FIG. 14 is a flow diagram which illustrates, schematically, a subprocess of the flow diagram of FIG. 11, for measuring infused volumes and performing drug concentration and infusion rate calculations with reference to a PKPD model;

FIG. 15 is a flow diagram which illustrates, schematically, an exemplary procedure for use of an infusion system such as that shown in FIGS. 2 and 8; and

FIG. 16 illustrates a simplified schema of an exemplary computing device in which various aspects of the disclosure may be implemented.

### DETAILED DESCRIPTION WITH REFERENCE TO THE DRAWINGS

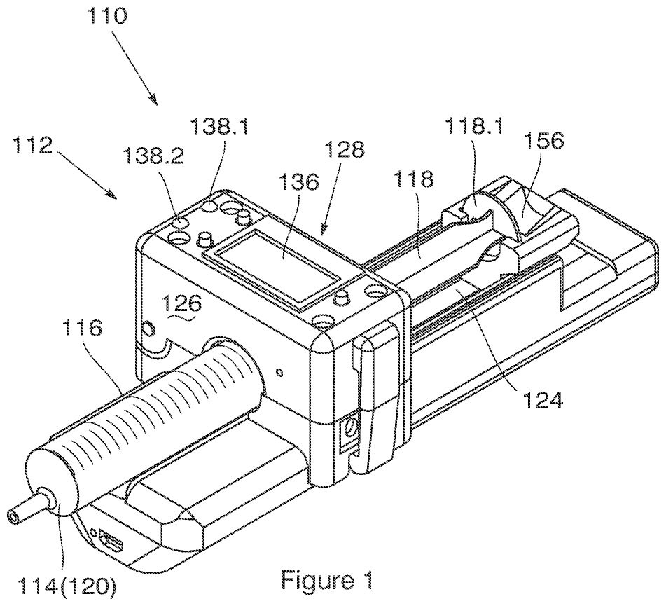

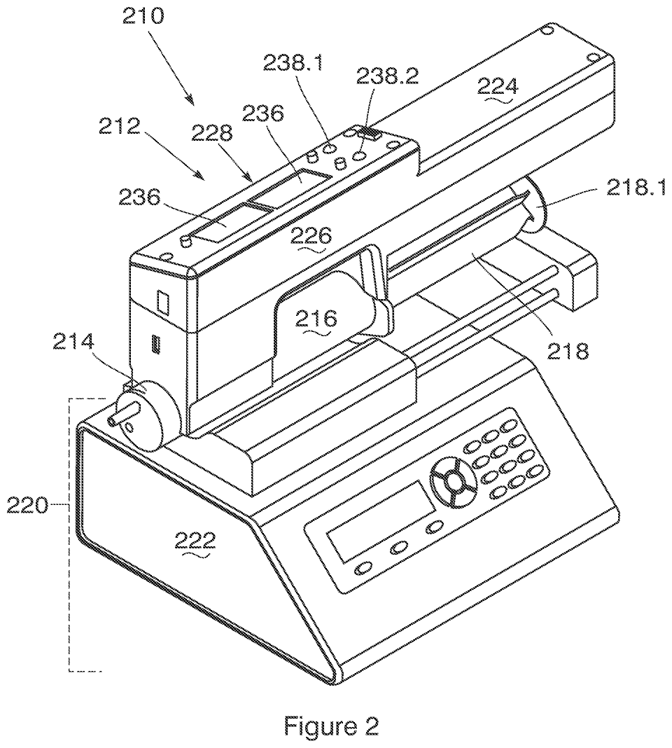

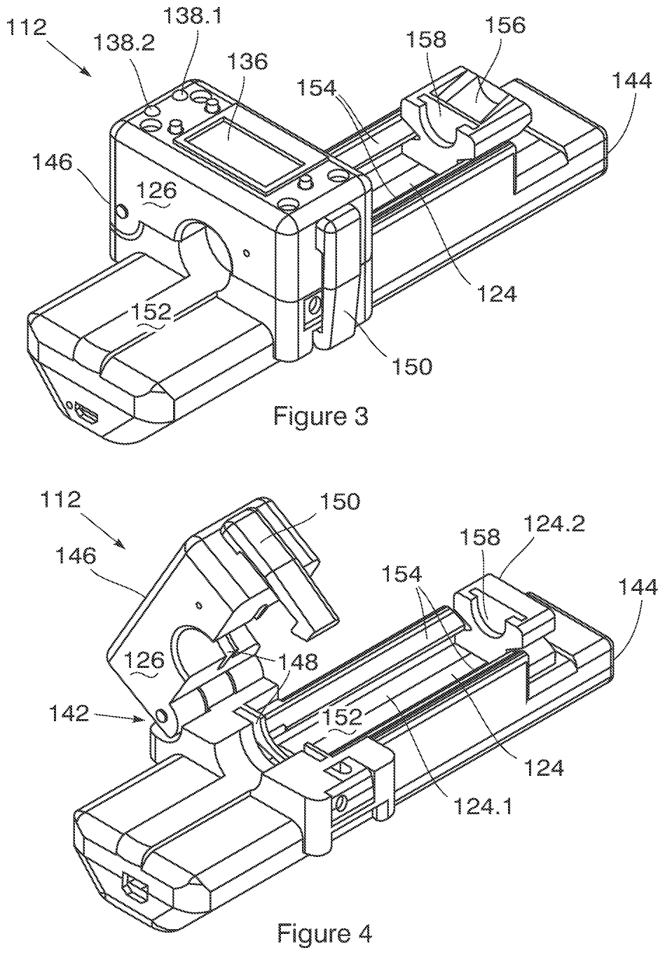

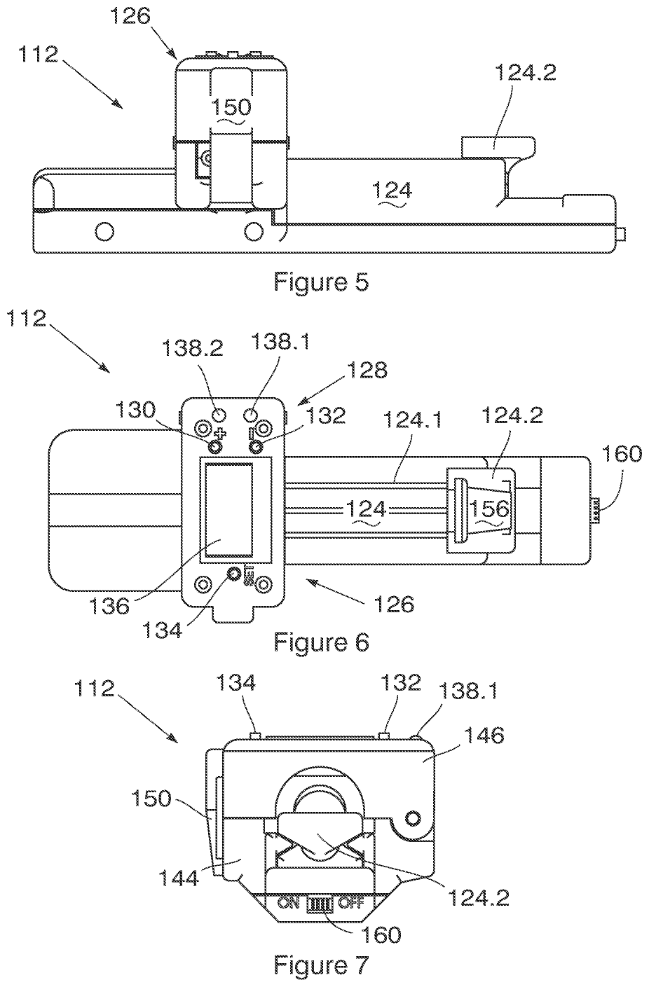

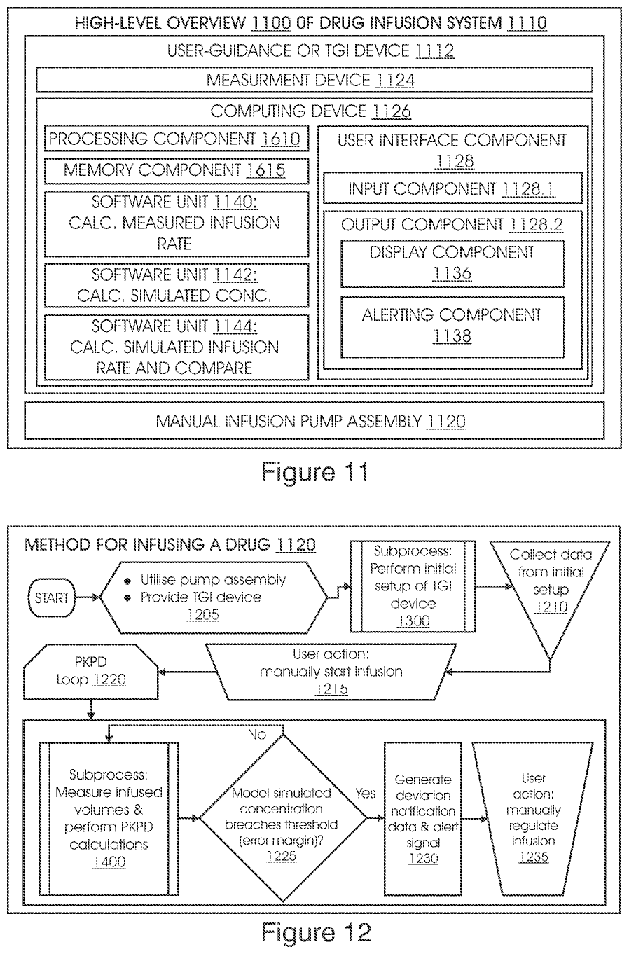

Referring to the drawings, FIGS. 1 to 10 show two exemplary variants (110; 210) of a drug infusion system for conducting manually-controlled infusion. FIG. 11 is a block diagram which provides a high level schematic overview (1100) of a basic variant (1110) of the system.

The system (110; 210; 1110) is intended to provide information and guidance to a user such as a clinician conducting a manual infusion procedure. (The word “conducting” in this context is to be understood broadly to encompass both performance and supervision of an infusion procedure.)

The system may be suitable for use by anaesthetists and sedationists performing anaesthesia or sedation with intravenous anaesthetic agents. It may be useful for infusion of an anaesthetic agent into a patient at a target concentration in a body compartment such as the patient's blood plasma or at an effect-site such as their brain, spinal cord or central nervous system.

The system described herein is not configured for making automatic adjustments to infusion rates. However, the system may permit standard syringes and zero-order, non-pharmacokinetic (non-PK) syringe pumps to be used to achieve PK-influenced infusion outcomes that are comparable to those which can be achieved with automatic, target-controlled infusion (TCI) devices. Zero-order pumps are able to drive a syringe plunger into a syringe barrel at a rate that can be set manually and which remains constant until a further manual adjustment is made. They lack functionality to interrogate PKPD models and to adjust infusion rates automatically, however. Since control of the overall infusion procedure and the rate of infusion remain at all times under the direct and necessary agency of the user instead of being delegated to an automatic TCI device, it may be expected that regulatory hurdles to be overcome by the described system may be less stringent than those applicable for TCI devices. This may in turn permit the systems and devices described herein to be more frequently upgraded as future PK models are developed. Costs to the end-user may also be expected to be reduced.

The system described herein may include a user-guidance device (112; 212; 1112), for which the term target-guided infusion (TGI) device has been coined. This term is used to distinguish the TGI device from target-controlled infusion (TCI) devices, since TGI devices do not offer automatic control of the infusion rate. TGI devices are intended only to guide, inform or advise users in their decisions relating to an infusion procedure. The information which a TGI device may provide can be used by a clinician to increase the adequacy of anaesthesia or sedation, to increase safety, and more accurately to predict emergence so that the time taken to wait for recovery from anaesthesia may potentially be reduced.

A clinician using the described system may select a drug from a list and administer it by hand from a syringe or with the assistance of the zero-order syringe pump. The system is able to track the volume and rate of infusion, and to calculate drug concentrations within the patient and required infusion rates. Near real-time mathematical calculations of PK and PD properties can be performed based on the quantity of drug given.

The TGI device may accordingly be useful for informing manually-controlled infusion of at least one drug from at least one manual drug infusion pump assembly into a patient.

More specifically, the TGI device may be useful for informing a required change to a pump volume displacement parameter of a manual drug infusion pump assembly, for use with manually-controlled infusion of at least one drug into a patient.

The TGI device may comprise at least one measurement component arranged to measure the pump volume displacement parameter; and a computing device arranged to calculate a measured rate of infusion of the drug into the patient based on the measured pump volume displacement parameter; and to calculate with reference to said measured rate of infusion and to at least one model selected from the group consisting of pharmacokinetic and pharmacodynamic models, a model-simulated drug concentration in the patient.

The computing device may further be arranged to derive user-guidance information based on the model-simulated drug concentration. It may be arranged to output the user-guidance information.

An advantageous feature of the infusion system described herein is that it may be adapted to administer multiple drugs concurrently or simultaneously, and to enable manual infusion adjustments to be made by the user based on calculations relating to interactions between the drugs. Currently available target-controlled infusion (TCI) systems generally do not take account of drug interactions.

Referring now to the drawings, the drug infusion system (110; 210; 1110) typically includes one or more standard syringes (114; 214) from which the drug or drugs are dispensed. A syringe typically has a barrel (116; 216) into which a plunger (118; 218) may be driven by applying force to a plunger flange (118.1; 218.1), either by hand or with the aid of an infusion pump.

FIG. 1 shows a first exemplary handheld variant (110) of the system. This variant may comprise a user-guidance or TGI device (112) and a manual pump assembly (120; 1120) in the form of a syringe (114).

FIG. 2 shows a second variant (210) of the drug infusion system. In this variant the drug is pumped from a syringe (214) by a non-PK, zero-order syringe pump instead of being manually injected by the clinician.

The variant (210) of the system is similar to the first-illustrated variant (110) insofar as it too comprises a user-guidance or TGI device (212) and a manual drug infusion pump assembly. However, in this variant the TGI device is configured to be attachable to the zero-order syringe pump. The pump assembly (220; 1120) of the second variant is provided by a combination of two cooperating parts, namely:
the syringe (214); and
a zero-order syringe pump (222) having a drive unit (not shown) for driving the plunger (218) of the syringe (214).

The TGI device (112; 212; 1112) may include a measurement component (124; 224; 1124) arranged to measure a pump volume displacement parameter. The volume displacement parameter is typically a reference characteristic or feature of the pump assembly which permits dispensed drug volumes to be measured, thereby enabling the amount of drug given to be calculated. The displacement parameter is advantageously selected such that variations in its value over time correlate to the rate of infusion of the drug. The reference characteristic used by the described embodiments is the linear distance travelled by the syringe's plunger (118; 218), which correlates in a generally linear relationship with the volume of drug infused.

To measure the distance of travel of the plunger, the measurement component (124; 224) may include a linear displacement sensor in the form of a rectilinear potentiometer (124.1; 224.1) or rheostat, slidably engageable with a cooperating slider (124.2; 224.2). The slider may be configured to hold or engage with a flange (118.1; 218.1) of the syringe plunger (118; 218) so that, in use, the slider may move in sympathy with the flange, thereby tracking changing positions of the flange along the longitudinal axis of the syringe barrel (116; 216) and the potentiometer. This in turn allows measurement, by the potentiometer, of the linear travel of the plunger and consequently the volume of drug delivered by the syringe. Other possible sensors may be used for measuring linear travel distance, such as range-finding means.

It will be appreciated that the pump volume displacement parameter may be a parameter other than plunger travel distance. It may comprise a rate of flow of the drug from the syringe, measurable with a flow meter. Thus, a flow meter may be provided instead of, or in addition to, the potentiometer.

The TGI device typically includes a computing device (126; 226; 1126) arranged to perform near real-time mathematical calculations based on relevant pharmacokinetic and pharmacodynamic properties and on the amount of drug given. The computing device may be arranged to calculate:
a measured rate of infusion of the drug based on the pump volume displacement parameter (e.g. the linear travel of the plunger) as measured by the measurement component; and
model-simulated or predicted drug concentrations based on the measured rate of infusion and at least one model selected from the group consisting of pharmacokinetic and pharmacodynamic (PKPD) models.

The computing device may be arranged to derive user-guidance information based on said model-simulated drug concentration; and to output the user-guidance information. The user-guidance information may be as previously described herein.

The computing device may be arranged to generate deviation notification data if the model-simulated or predicted drug concentration, calculated with reference to the measured rate of infusion and the model, breaches a definable threshold concentration (that is, if the calculated concentration as predicted by the model differs from a target concentration by more than a pre-selected error margin).

The computing device (126; 226; 1126) may be arranged to perform additional calculations to establish required infusion rates for attaining and maintaining drug concentration at pre-selected levels. The computing device may thus be arranged to calculate, with reference to the pump volume displacement parameter and the PKPD model or models, a model-simulated infusion rate required for achieving a drug concentration within a target concentration range. These types of calculations may typically be expected to be provided by the variant (210) and the results may be consulted by the clinician or an assistant to set or periodically adjust the zero-order (constant) rate of infusion delivered by the pump.

The computing device may further be arranged to compare the measured rate of infusion against the required infusion rate, thereby to establish a deviation of the measured infusion rate from the required infusion rate; and to generate deviation notification data if the deviation breaches a definable threshold (that is, if the deviation from the required infusion rate, whether above or below it, exceeds a pre-selected error margin).

The computing device may be arranged to execute a feedback algorithm capable of repeatedly updating its calculations in response to variations in the parameters such as the measured rate of infusion and elapsed time.

The computing device may be integrated as part of the TGI device or it may be provided by an external device such as a smartphone or other mobile computing device. A purpose-created software application may be installable on the computing device.

In exemplary embodiments not shown in the drawings, the user-guidance or TGI device may have a spatially distributed arrangement whereby the measurement component and at least a portion of the computing device may be provided separately from one another. The computing device and the measurement component may be connectable to each other by means of suitable wired or wireless data communication means.

A spatially distributed arrangement of this type may comprise:
a remote, non-attached or non-attachable computing device such as, without limitation thereto, a cellular telephone, tablet computer or laptop computer; and
a sub-component of the computing device attachable to the syringe or syringe pump, which is configured to perform limited computing functions restricted to measurement of the displacement of the syringe plunger and transmission of the measurement information to the remote, non-attached or non-attachable computing device.

A spatially distributed arrangement of this type may be advantageous for the following reasons:
Updates to software may more easily be carried out via the Internet by replacement of files. This may reduce the need to recall TGI devices for software updates, which may otherwise be required if all computing functionality is placed on the attachable portion of the TGI device.
It may be feasible to provide greater computing power and larger internal memory on a non-attachable or non-attached computing device than on an attachable sub-component of the TGI device.
By comparison with the display screen on an attachable sub-component, it may be feasible to provide a display screen on the non-attached or non-attachable device that is larger, has better resolution and is in colour.

The TGI device (112; 212; 1112) may include a user interface (UI) component (128; 228; 1128). The UI component may comprise an input component (1128.1 in FIG. 11) and an output component (1128.2 in FIG. 11).

The input component may be arranged to receive values representative of the pump volume displacement parameter, e.g. the linear distance travelled by the syringe plunger, as well as other pre-stored or user-provided input information pertinent to the infusion.

The output component may include a display component (1136 in FIG. 11) with one or more displays (136; 236) for displaying calculated and updated results, input parameter values and other pertinent information. Without limitation thereto, the information which may be displayed may be selected from the group consisting of:
simulated or predicted drug concentrations in the patient's blood plasma (Cplasma or Cp);
simulated or predicted drug concentrations at effect-sites (Ce) such as the patient's brain, spinal cord or central nervous system;
simulated or predicted infusion rates required to maintain selected target blood concentrations and/or to achieve selected clinical effects, as well as updates and changes thereto;
required infusion pause durations;
bolus doses required to achieve target concentrations;
times to next bolus;
clinical effects, for example the probability of:
   non-response to verbal command; and/or
   non-response to varying degrees of painful stimulation (e.g. skin incision, laryngoscopy, etc.);
other clinical effects such as:
   BIS (bispectral index) or other indices of electroencephalographic changes; and/or
   clinical end points such as time to offset of drug effect (for example, time to recovery of consciousness); the end points may be calculated with reference to pre-selected recovery concentrations; and
drug interactions regarding clinical effects.

Future research may be expected to explore models providing data relating to new clinical end points. In such circumstances it will be appreciated that the system and variants of the TGI device described herein may be upgraded to take account of the data available from such further models, and to be configured to provide guidance to users regarding infusion rates required for achieving such new and additional clinical end points.

The information may be displayed in raw form or as graphical representations.

The TGI device (112; 212; 1112) may be configured to alert the user to avoid over- or under dosing. The device may thus be configured to alert the user at clinically feasible timing intervals to change infusion rates. Alerts may be generated in near real-time (e.g. within seconds or milliseconds from the occurrence of the events to which they relate).

The output component (1128.2) may thus include at least one alerting component (1138) configured to produce at least one user-discernible alert or alarm signal responsive to generation of the deviation notification data. Visual means such as lamps may be provided for this purpose. Instead or in addition, the alerting component may include means selected from the group consisting of audio, haptic and print means. It may include any other suitable means for drawing a user's attention. The alerting component may serve to alert the clinician that manual setting or adjustment of the operation of the manual drug infusion pump assembly is required to adjust the rate of infusion, and/or that a bolus must be administered.

In the embodiments of the system (110; 210) shown in the drawings, the TGI devices (112; 212) are stand-alone devices of unitary construction.

They may be configured to be engageable with and detachable from their respective manual drug infusion pump assemblies (114; 222), preferably although not necessarily in a tool-free manner.

Preferably the TGI devices may be configured such that they will not interfere with the normal functioning of syringe pumps to which they may be attached; nor interfere with access to the control systems of such pumps.

Engagement formations may be provided on the TGI device. Such formations may include retaining slot formations, clamping arrangements, clips, resiliently-biased catches, over-centre fastening arrangements, stop formations with complementary receiving formations, formations defining surfaces arranged to engage the pump assembly in a friction fit or an interference fit, elastic bindings, manually-operable fasteners such as thumbscrews, wingnuts, etc. The engagement formations may advantageously be configured for complementary engagement with formations present on the infusion pump and/or the syringe with which a particular TGI device is designed to be used. Interchangeable fitment means having different types of engagement formations may be provided to permit a TGI device to be engaged with a variety of different types of syringes and infusion pumps. Preferably the engagement formations may be configured such that they will not interfere with the normal functioning of syringe pumps with which they may engage; nor interfere with access to the control systems of such pumps.

Calculations relating to drug concentrations and predicted or simulated infusion rates may be made by utilising PK and PD mathematical models available in the scientific and clinical literature. An exemplary selection of publications is cited in the section entitled “References” at the foot of this description. Typically, more than one drug concentration may be calculated, e.g. both Cp and Ce.

The input component (1128.1) may include three push-buttons (FIG. 6: 130, 132, 134, and FIG. 9: 230, 232, 234). In the embodiments shown in the drawings, two of the push buttons may be provided for range adjustment, i.e. for plus (+) (130, 132) and minus (−) (230, 232) value adjustment, whilst the third button (134; 234) may provide a “set” function enabling a user to set a value once they have selected it using the first two buttons. It will be appreciated that a wide variety of other input devices, such as those described elsewhere herein, may be implemented. A touch-sensitive screen displaying menu items and icons may be used instead of, or in addition to, the push-buttons.

The output component may include a graphical user interface (GUI) which may comprise one or more displays (136; 236). In use, the displays may display the types of information required for input by the user, values entered by the user, and the results of calculations and comparisons performed by the computing device, as well as updates thereto. The displays (136; 236) may comprise one or two 1.3-inch OLED display modules, depending on the variant of the device.

The alerting component (1138) may comprise a blue LED lamp (138.1; 238.1) and an amber LED lamp (138.2; 238.2). The computing device may be configured to illuminate the lamps when the deviation notification data is generated.

The system may be configured such that the blue LED lamp is illuminated when the targeted effect-site or plasma concentration (as simulated by the model) is below the desired level by an amount which exceeds the error margin. The system may be configured such that the blue LED lamp is illuminated when the measured infusion rate is below the required infusion rate by an amount which exceeds the error margin.

The blue lamp may thus serve to alert the clinician that the error margin has been breached and prompt him or her to increase the infusion rate, either by manually administering a further bolus dose of the anaesthetic agent or, if a syringe pump is being used, by manually increasing the constant rate of infusion administered by the pump. The system may be configured to suggest a required bolus dose and/or increased infusion rate to be administered as calculated according to the state of the pharmacokinetic model.

The system may be configured such that the amber LED lamp is illuminated when the targeted effect-site or plasma concentration (as predicted by the PK model) rises above the desired level by an amount exceeding the error margin. The system may be configured such that the amber LED is illuminated when the measured infusion rate rises above the required infusion rate by an amount exceeding the error margin.

The amber LED lamp may accordingly serve to alert the clinician that the error margin has been exceeded and prompt him or her to cease infusion for a pause duration which may be calculated by the TGI device and displayed.

The system may be configured such that, when a simulated concentration (Cp or Ce) has reached the targeted concentration, the system may calculate and suggest a reduced zero-order infusion rate required to maintain the Cp or Ce at the targeted concentration.

In the described embodiments, if the TGI device is on target both LED lamps will be switched off.

Embodiments of the TGI device may be configured to function in a non-targeting mode wherein simulated concentrations are displayed but not compared against definable thresholds. When in such a mode, both LEDs will also be switched off.

The error margin selectable by the user may typically be expected to range from 5% to 30%. If, say, an error margin of 30% has been selected in respect of a 2 μg/ml target concentration for a given infusion, the user will be alerted if the measured rate of infusion deviates by 30% or more from a model-simulated or predicted infusion rate required to achieve a 2 μg/ml target concentration. Although error margins outside the range of 20% to 30% are feasible, margins exceeding 30% may be considered to be clinically unacceptable and margins lower than 10% may result in unnecessarily frequent alerts.

As best seen in FIGS. 1, 3 and 4, the handheld variant of the TGI device may comprise a body (142) which includes first and second, hingedly or pivotably connected body portions. These body portions may comprise a base member (144) and a closure member (146). During use of the handheld variant, the base member and closure member may be closed over the syringe to secure it. Engagement of the hinged body (142) with the syringe can achieved by opening the body and mounting the syringe on the base member (144). Engagement formations defining slot formations (148) may be provided in the base member, with which the syringe's barrel flange may be complementarily engageable.

The base and closure members may be snap-lockingly engageable with each other. For this purpose, resiliently-biased clasp or catch formations (150) may be provided. During use of the handheld TGI device, the closure member may be snap-lockingly closed over the syringe and the base member to lock the syringe in place.

In other embodiments (not shown) the base and closure members may be provided as discrete parts which may be releasably locked to each other over the syringe.

The base member (144) may define a syringe bed or channel (152) for holding the syringe. Guide means in the form of opposed elongate rails (154) may be provided, laterally spaced from each other on opposed sides of the syringe bed. The rails may be configured to be engageable with complementary formations defined in the slider (124.2) so that the slider (124.2) may be held slidably captive within the syringe bed.

A thumb grip (156) may be provided as part of the slider (124.2) of the measurement component (124), to facilitate manual driving of the slider and syringe plunger by the thumb of a user along the length of the potentiometer (124.1). The thumb grip may define a slot formation (158) for engaging with the plunger flange (118.1).

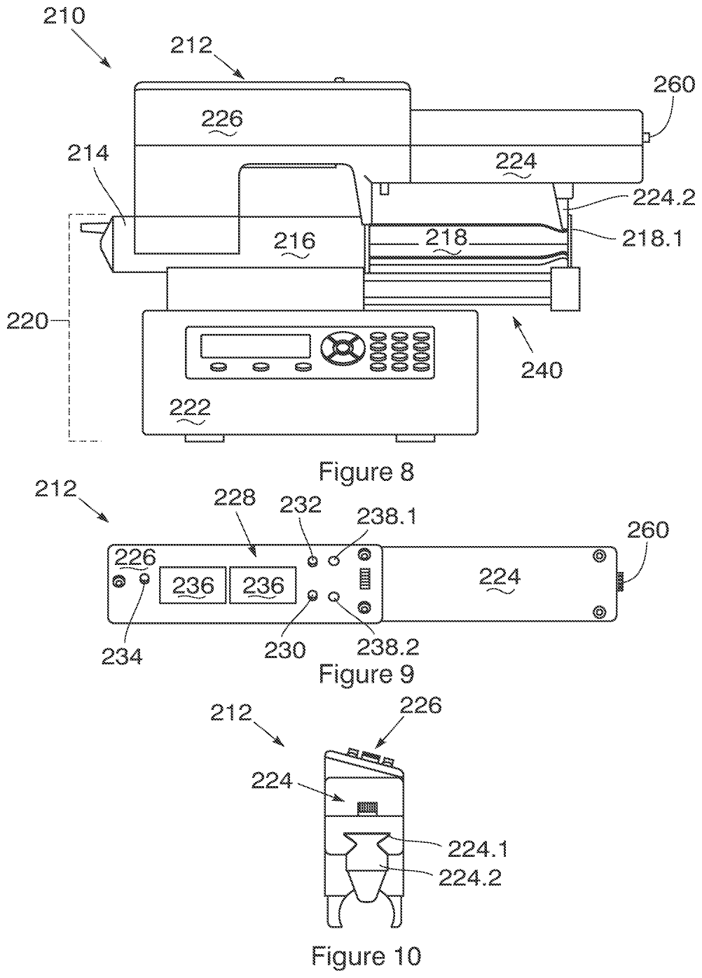

As shown in FIG. 8, certain types of non-PK infusion pumps (222) may include a movable carriage (240) for driving the syringe plunger. In such cases the measurement component (224) may advantageously be configured for engagement with the infusion pump in proximity to the syringe. This may permit the plunger flange (218.1) to be engaged with the slider (224.2) of the measurement component so that it may track linear displacement of the plunger as the carriage drives the plunger into the barrel.

Certain embodiments of the TGI device may be adapted to be secured to the pump assembly in a fixed condition. Fastening means such as screws, grub-screws, bolts, clamps, adhesives, bindings, and the like may be used for fixing the TGI device to the assembly in more permanent arrangements of this nature.

During operation of a TGI device, once the clinician has commenced the infusion process, near real-time calculations are performed by the microprocessor component and displayed by the output component's display screen or screens (136; 236). This keeps the user informed of drug concentrations and clinically relevant information.

As previously mentioned, certain embodiments of the system (not shown) may be arranged to monitor infusion of two or more drugs concurrently or simultaneously. Simultaneous delivery of drugs occurs commonly during intravenous anaesthesia since it can bring forth supra-additive or synergistic interactions which can reduce required drug quantities and improve recovery times. Multiple anaesthetic agents having fast onset and offset times are useful for balancing adequate hypnosis or analgesia with rapid recovery.

In embodiments adapted for the infusion of multiple drugs, the system may comprise a plurality of drug infusion pump assemblies and an interactive network of measurement components, one measurement component being provided in association with each pump assembly. The measurement components may be arranged in data communication with the computing device.

The computing device may be arranged to consult drug-interaction protocols and to calculate information relating to drug interactions. This information may be used in combination with simulated patient concentrations (Cp or Ce) to predict clinically relevant end points and required infusion rates based on the interactions.

Embodiments of this type may comprise a plurality of TGI devices. However, since only one computing device is required to monitor multiple measurement components and to conduct processing, a computing device of a first TGI device may be established as a master and that of second and further TGI devices as clients or slaves of the master.

Also, in certain embodiments of the system a single TGI device may be provided which includes a plurality of pump assemblies, each paired with a corresponding measurement device.

Wireless or wired communication technology may be employed to enable the multiple measurement components and the computing device to communicate with one another. Wi-Fi® technology following a TCP/IP or MQTT protocol may be implemented to provide the wireless communication.

A dual-core microprocessor may be advantageous for incorporation in the computing device or devices, with one core being provided for conducting mathematical calculations and the other for conducting communications between the master and its client/s. By way of example, the microprocessor used may comprise an ESP32 processor available from Espressif Systems.

To set up systems catering for multiple drug infusions, the required concentration for a first of the drugs may be set by the user. The system may then make calculations for infusion of the other drug or drugs in relation to the first, and to keep the user updated on when to activate the various infusion pumps (or to depress the syringe plungers manually) to take account of the model-simulated concentrations and required infusion rates. In some cases the system may additionally advise the clinician regarding required bolus volumes to be delivered from each pump in order to achieve the required end point.

Currently available PK models allow for drug interactions to be predicted for no more than three drugs. In more complex anaesthetic procedures, however, up to five separate anaesthetic agents may be administered concurrently by clinicians. Procedures of such complexity cannot at present be mediated by PK models. However, future research and progress in the field may be expected to deliver PK and PD models capable of simulating and predicting the interaction of greater numbers of drugs. By way of example, if a model becomes available for five drugs then the described system may be expanded to include five separate pump assemblies matched with five separate measurement components connected to one or more computing devices by wired or wireless means.

The described TGI devices may each include a separate button or sensor switch which may act as a syringe presence sensor configured to detect syringe removal and insertion. Typically, a standard 10 ml, 20 ml or 50 ml syringe may be used with a TGI device, but it will be appreciated that TGI devices may also be configured for use with other suitably-sized syringes.

Embodiments of the TGI device may be powered by means of a battery of galvanic cells (e.g. lithium-polymer cells) chargeable via a USB-B cable and a battery-charging module (not shown). Power to the TGI devices may instead or in addition be provided directly by a mains supply or other suitable power source such as motorised or manually cranked (“wind-up”) or wind-powered generator, or solar panels. On-off switches (160; 260) and charging means (not shown) may be provided on the TGI device.

The TGI devices may include voltage regulators (not shown) to maintain constant voltage across the, or each, microprocessor and to ensure accurate volume displacement readings from the measurement component. Voltage dividers may be included to measure the battery voltage and alert the user if it drops. The TGI device may be configured to check the battery periodically for appropriate voltage and to warn the user to connect to a charger if needed. Also, in variants of the system involving infusion pumps, the TGI device may be configured to prevent initialisation of the infusion pump if the battery voltage is too low, and to display a warning in this regard.

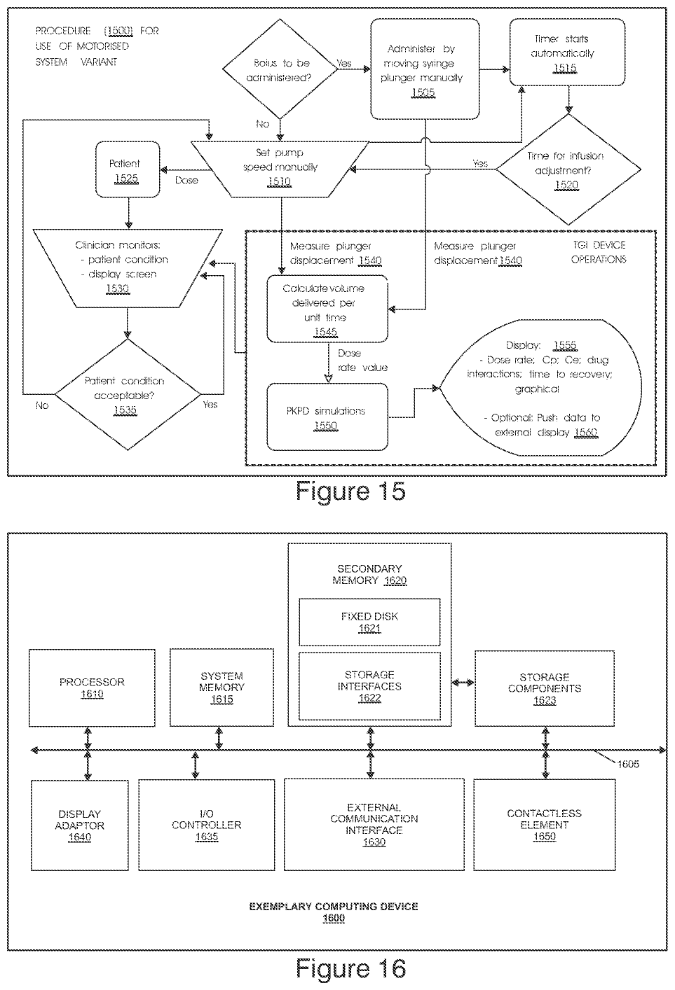

The system (110; 210; 1110) and the TGI device (112, 212; 1112) described above may implement a method for infusing a drug into a patient. Referring to FIGS. 12 and 15, examples of the method are illustrated by flow diagrams (1200; 1500).

The method (1200) may include:
utilising (1205) a manual drug infusion pump assembly (120; 220)—either a syringe or a combination of a syringe and a syringe pump—the assembly having a pump volume displacement parameter;
providing a user-guidance or TGI device (112; 212; 1112) as herein described;
performing (1300) an initial setup of the TGI device, including inputting syringe dimensions;
collecting (1210) data from the initial setup;
manually starting infusion (1215) of the drug into the patient using the syringe or the combination of the syringe and pump (depending on the variant of the TGI device used); and
operating the TGI device's computing device to execute a loop (1220) involving the following steps:
   executing a subprocess (1400) to calculate a measured rate of infusion of the drug into the patient based on the pump volume displacement parameter; and to calculate and update with reference to the pump volume displacement parameter and one or more PK or PD models, a model-simulated drug concentration in the patient;
   checking whether the model-simulated drug concentration breaches a definable threshold (1225), e.g. a percentage deviation from a definable target concentration;
   generating (1230) deviation notification data and an alert signal if the concentration breaches the threshold; and
   manually regulating or adjusting (1235) the infusion rate responsive to the alert signal.

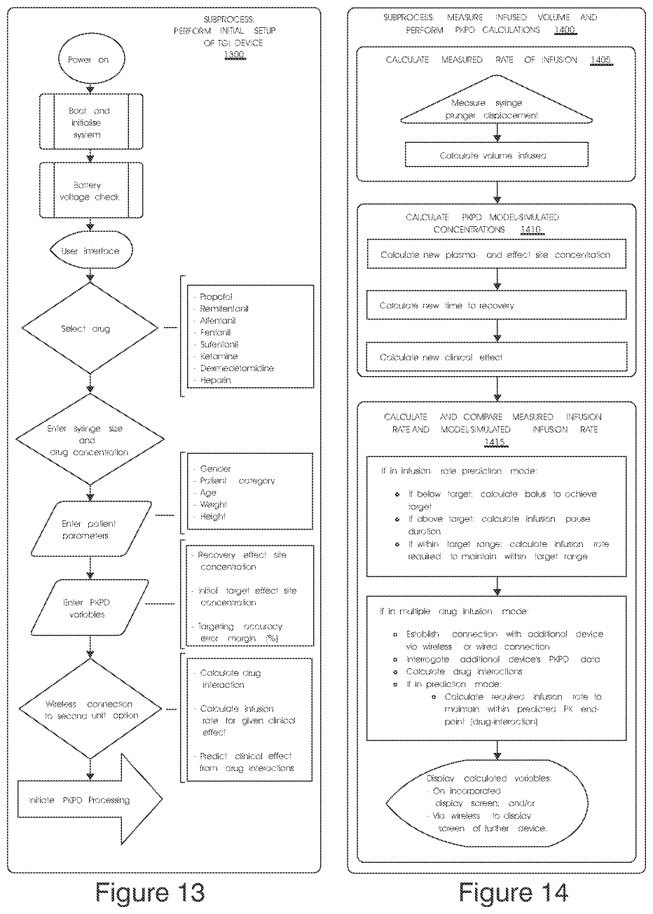

FIG. 13 shows steps involved in the subprocess (1300) for performing the initial setup of the TGI device. The user may commence the setup by providing power to the device and booting and initialising it, after which the device may conduct a battery voltage check and periodically repeat it. Based on the results of these checks the TGI device may provide warnings to the user to connect to a charger if necessary. In variants of the system involving an infusion pumps, the TGI device may also prevent initialisation of the pump if the battery voltage is too low.

The clinician may then decide upon the drug or drugs to be used. If more than one is to be used, multiple TGI devices may need to be prepared. If an external user interface is to be used, input and output can be set up to take place via an external device such as a mobile phone, laptop computer, etc. Wireless communication, such as by Wi-Fi® or Bluetooth®, can be established between the external device and the TGI device. If an external user interface will not be used then input and output may take place via the TGI device.

The computing device (126; 226) may execute an input algorithm which interrogates the user or a pre-loaded memory component, in order to obtain input parameters selected from the group consisting of:
Anaesthetic agent/s or drug/s to be used (default: propofol). A menu offering a selection of drugs may be presented to the user. In certain embodiments the selection may include propofol, remifentanil, alfentanil, fentanyl, sufentanil, ketamine, dexmedetomidine and heparin.
Nominal syringe size and/or volume (default: 20 ml).
Concentration of drug to be infused (default: fixed). An alternative, “calculated” option may be offered, whereby a routine can be executed to interrogate the user to enter the quantity of drug added to the syringe (default 100 mg for a 20 ml syringe).
Patient parameters such as gender, patient category (neonate, child, adult), age, weight and height.
PKPD model to be used (selectable from a database).
PKPD variables and targets selected from the group consisting of:
   initial concentration at effect-site (default: none);
   target plasma concentration (Cp);
   target effect-site concentration (Ce), e.g. a target brain concentration to achieve a specific clinical end point such as loss of consciousness;
   recovery (emergence) concentration (default: 1.3 μg/ml); and
   effect level information for a drug effect to be achieved in the patient.
Whether other drugs (e.g. opioids) will be administered synergistically so that drug interactions can be taken into account (default: “no”).
Maximum targeting accuracy error margin (default: 10%).

The effect level information may be qualitative or quantitative and may relate to the nature of a specific clinical effect or end point sought, such as induction, loss of consciousness, non-responsiveness to surgical stimulus, depth of anaesthesia, time to eye-opening, emergence, etc. The effect level information may include at least one effect level value within a predetermined effect scale. The information may be selectable from a menu or list offered by the user interface component.

The effect scale may be a selectable scale based on a percentage probability of the patient responding during a surgical stimulus. Instead or in addition, the effect scale may be based on the BIS number, which is a numerical value usually obtained from processed electroencephalographic (EEG) monitoring. It denotes a patient's “depth of anaesthesia”. Typically, a BIS number of 0 indicates extremely deep anaesthesia while a BIS number of 100 indicates a fully awake condition of the patient. Certain embodiments of the TGI device may be configured to predict infusion rates based on a selected BIS number.

A data connection to a second or further TGI device may be established if concurrent infusion of a second or further drug is required. In use, if two or more TGI devices are simultaneously delivering agents, this will result in drug interactions. These are calculated and taken into account by the TGI devices.

The user may engage or couple the TGI device with the infusion pump assembly, whether that be the syringe or the combination of syringe pump and syringe. Depending upon the particular design and configuration of the TGI device and the pump assembly, engagement of the TGI device may involve attaching, fitting, clamping, securing or fixing the TGI device to the pump assembly.

The user may be prompted by the computing device to calibrate the TGI device by entering further input parameters. Thus, the input algorithm may interrogate the user to calibrate a “full” position of the syringe (the plunger is drawn to its starting division or gradation mark and the user presses the “set” button (134; 234)) and an “empty” position (the plunger is depressed to its ending division or gradation mark and the user again presses the “set” button). After calibration, the user may demount, disengage or decouple the syringe from the TGI device or the non-PK pump, fill it with the applicable drug and remount it.

Certain embodiments of the TGI device may avoid the need for manual calibration of the syringe. In such embodiments the computing device may provide menus (or other means) to allow the user to select a specific syringe make and model from a database. The database may contain specific information required by the microprocessor to accurately calculate syringe displacement for the specific syringe selected by the user.

PKPD processing may then be commenced. The computing device may calculate a dose regimen and infusion requirements for achieving a steady state and/or the selected concentrations, effects or end points. Starting settings of Ce, Cp, and BIS may be displayed on the displays (136; 236). The computing device may cause the blue LED alert lamp (138.1; 238.1) to be illuminated to serve as a “below target” alert. This indicates to the user that infusion of the drug is required.

Referring to FIG. 14, the subprocess (1400) for measuring infused volume/s and performing the PKPD calculations may include any or all of the following steps:
Calculating a measured rate of infusion based on varying values returned by the measurement components as it measures the pump volume displacement parameter (1405). This step may involve measuring the syringe plunger displacement and calculating the volume infused. A software unit (1140 in FIG. 11) executable by the computing device may be provided for carrying out these calculations.
Calculating PKPD model-simulated or predicted concentrations (1410). This step may involve calculating a new plasma and effect-site concentration, calculating a new time to recovery and calculating a new clinical effect. A software unit (1142 in FIG. 11) executable by the computing device may be provided for carrying out these calculations.
Calculating and comparing the measured infusion rate and a model-simulated, required infusion rate (1415). A software unit (1144 in FIG. 11) executable by the computing device may be provided for carrying out these calculations and comparisons. This step may include the following sub-steps:
   If the TGI device is configured for infusion rate prediction, calculating any of the following:
      bolus volumes required to achieve the target concentration if the concentration is below target;
      an infusion pause duration if the concentration is above target of if a pause is required to allow for equilibration as the infused drug mixes with the patient's blood and is transferred from the patient's plasma to their brain; and/or
      if the concentration is within the target range, an infusion rate required to maintain the concentration within the target range.
   If the TGI device is combined with another TGI device in multiple drug infusion mode, this step may include establishing communication with the other device by wireless or wired means, interrogating the PKPD data of the other device and calculating applicable drug interactions. The step may also involve calculating required infusion rates to maintain concentrations within a predicted PK end point or end points based on the drug interactions.
   Displaying calculated values and variables.

The clinician may then commence manually controlled infusion of the drug or drugs.

The TGI device calculates the initial bolus required, allowing the clinician to administer the correct amount to avoid overshoot of the specific target. Once the clinician has administered the amount, the TGI device will calculate the duration of pause required for the drug to equilibrate (achieve clinical effect). Thereafter the TGI device will calculate and display the infusion rate required to maintain the current target.

The clinician continues to infuse the drug until the TGI device generates an alert to notify the user that its calculated plasma or effect-site concentration, based on the volume of drug dispensed from the syringe, exceeds the PK model-simulated target concentration by more than the pre-selected error margin, e.g. by more than 30%. In the embodiments shown, this alert is a visual alert provided by the amber LED lamp (138.2; 238.2).

At any point the clinician can choose to adjust the target up or down, or to change the clinical end point or the drug interaction, after which the TGI device will recalculate the infusion rate and alert the clinician appropriately. The TGI device will alert the clinician in a timeous manner when to adjust the infusion rate to maintain the target within the error margin. The narrower the error margin, the more regular the notifications will be. If the clinician ignores the alerts, the TGI device will continue to monitor the amount of drug delivered while recalculating the aforementioned parameters. The alerts might be altered in an attempt to gain the clinician's attention (e.g. by louder audible alarms or flashing LED lamps).

FIG. 15 illustrates a procedure (1500) which a clinician using the variant (210) of the infusion system may follow. If a bolus dose is to be administered the clinician administers the drug by moving the syringe plunger manually (1505) or by setting the speed or rate of infusion of the pump manually (1510). A timer operated by the computing device may then automatically be started (1515). If the TGI device calculates (1520) that it is time for an infusion adjustment to be made, an alert signal may be generated prompting the user to manually adjust (1510) the speed or rate of infusion of the pump. Unless so adjusted, the infusion pump continues to deliver a constant dose to the patient (1525). The clinician may monitor (1530) the patient's condition as well as the display screens of the TGI device. If the patient's condition is deemed (1535) not to be acceptable, the infusion pump speed may again be manually adjusted (1510). These steps may be repeated as appropriate until the infusion procedure has been completed.

Meanwhile, the measurement component of the TGI device measures (1540) the plunger's displacement and the computing device calculates (1545) volume delivered per unit time based on these measurements, giving a dose rate value which can which inform repeated PKPD simulations (1550). Results of the simulations are output (1555) to the displays of the user interface component, for monitoring by the clinician. Optionally the results may be pushed (1560) to one or more external displays.

The foregoing description of the method is illustrative only and is not intended to limit the generality of the steps of the method and the order in which they may be performed. In particular, it will be appreciated that the order in which the input parameters may be entered, and the order which the user may perform the various steps may, if practically feasible, be changed without departing from the scope of the invention. Additionally, some of the steps are optional and need not necessarily be performed during use of all variants of TGI devices.

FIG. 16 is a simplified schema illustrating exemplary components of a computing device (1600), for implementing aspects of the method described with reference to FIGS. 12 to 15.

The computing devices (126; 226) of the described TGI devices (112; 212) may include components of the exemplary device (1600). The various participants and elements in the system block diagram of FIG. 11, may use any suitable number of subsystems or components of the exemplary computing device (1600) to facilitate the functions described herein.

The exemplary computing device (1600) may be embodied as either of the computing devices (126; 226), or as any other form of data processing device including a personal computing device (e.g. laptop or desktop computer), a server computer (which may be self-contained or physically distributed over a number of locations), a client computer, or a communication device such as a mobile phone (e.g. cellular telephone), satellite phone, tablet computer, personal digital assistant or the like. Different embodiments of the computing device may dictate the inclusion or exclusion of various components or subsystems described below.

The computing device (1600) may include subsystems or components interconnected via a communication infrastructure (1605) (for example, a communications bus, a network, etc.).

The computing device (1600) may be suitable for storing and executing computer program code.

The computing device (1600) may include a processing component comprising one or more processors (1610) for executing the functions of components described below, which may be provided by hardware or by software units executing on the computing device. At least some of the software units may be provided by one or more software applications installable and executable on the computing device. Instructions may be provided to the processor or processors (1610) to carry out the functionality of the described components.

The one or more processors (1610) may include one or more of: CPUs, graphical processing units (GPUs), microprocessors, field programmable gate arrays (FPGAs), application specific integrated circuits (ASICs) and the like. In some configurations, a number of processors may be provided and may be arranged to carry out calculations in parallel and/or simultaneously.

In some implementations various subsystems or components of the computing device (1600) may be distributed over a number of physical locations (e.g. in a distributed, cluster or cloud-based computing configuration) and appropriate software units may be arranged to manage and/or process data on behalf of remote devices. Aspects of the processing necessary for the system's functionality may thus be implemented or stored remotely.

The computing device (1600) may include at least one memory component (1615). The memory component or components may include system memory (1615), which may include read only memory (ROM) and random access memory (RAM). The memory component or components may comprise computer-readable media. The memory component or components may be arranged to store the software units, the input parameters required for operation of the system and/or values representative of results of calculations performed by the computing device (1600).

A basic input/output system (BIOS) may be stored in ROM. System software may be stored in the system memory (1615) including operating system software. The memory components may also include secondary memory (1620). The secondary memory (1620) may include a fixed disk (1621), such as a hard disk drive, and, optionally, one or more storage interfaces (1622) for interfacing with storage components (1623), such as removable storage components (e.g. magnetic tape, optical disk, flash memory drive, external hard drive, removable memory chip, etc.), network attached storage components (e.g. NAS drives), remote storage components (e.g. cloud-based storage) or the like.

The computing device (1600) may include an external communications interface (1630) for operation of the computing device (1600) in a networked environment enabling transfer of data between multiple computing devices (1600) and/or the Internet. Data transferred via the external communications interface (1630) may be in the form of signals, which may be electronic, electromagnetic, optical, radio, or other types of signal. The external communications interface (1630) may enable communication of data between the computing device (1600) and other computing devices including servers and external storage facilities.

In particular, the external communications interface may enable communication of data between the computing device (1600) and other computing devices associated with further TGI devices. It may enable communication of data between the computing device (1600) and measurement components associated with further TGI devices. A plurality of inter-communicating TGI devices may be employed for concurrent or simultaneous infusion of multiple drugs.

Web services may be accessible by and/or from the computing device (1600) via the communications interface (1630).

The external communications interface (1630) may be configured for connection to wireless communication channels (e.g. a cellular telephone network, wireless local area network (e.g. using Wi-Fi®, satellite-phone network, Satellite Internet Network, etc.) and may include an associated wireless transfer element, such as an antenna and associated circuitry.

The external communications interface (1630) may further include a contactless element (1650), which is typically implemented in the form of a semiconductor chip (or other data storage element) with an associated wireless transfer element, such as an antenna. The contactless element (1650) may be associated with (e.g. embedded within) the computing device (1600) and data or control instructions transmitted via a cellular or Wi-Fi® network may be applied to the contactless element (1650) by means of a contactless element interface (not shown).

The contactless element interface may function to permit the exchange of data and/or control instructions between computing device circuitry (and hence the cellular network) and the contactless element (1650). The contactless element (1650) may be capable of transferring and receiving data using a near field communications capability (or near field communications medium) typically in accordance with a standardized protocol or data transfer mechanism (e.g., ISO 14443/NFC). Near field communications capability may include a short-range communications capability, such as radio-frequency identification (RFID), Bluetooth®, infra-red, or other data transfer capability that can be used to exchange data between the computing device (1600) and an interrogation device. Thus, the computing device (1600) may be capable of communicating and transferring data and/or control instructions via a cellular network, Wi-Fi® network and/or near field communications capability.

The computer-readable media in the form of the various memory components may provide storage of computer-executable instructions, data structures, program modules, software units and other data. A computer program product may be provided by a computer-readable medium having stored computer-readable program code executable by the central processor (1610). A computer program product may be provided by a non-transitory computer-readable medium, or may be provided via a signal or other transient means via the communications interface (1630).

Interconnection via the communication infrastructure (1605) may allow the one or more processors (1610) to communicate with each subsystem or component and to control the execution of instructions from the memory components, as well as the exchange of information between subsystems or components.

Peripherals (such as printers, scanners, cameras, or the like) and input/output (I/O) devices may couple to or be integrally formed with the computing device (1600) either directly or via an I/O controller (1635). The peripherals may include or be selected from the group consisting of input push-buttons, alerting lamps (e.g. LED lamps), speakers, haptic devices, olfactory devices, mouse devices, touchpads, keyboards, microphones, displays, etc.

A display or video adaptor (1640) may be provided for connecting the displays (136; 236; 1136) to the processor or processors (1610). The displays may be touch-sensitive displays and may be coupled to or integrally formed with the computing device (1600).

### Utility of the TGI Device for Pharmacokinetic and Pharmacodynamic Investigations

Although the primary purpose of the disclosed TGI device is to provide clinicians with predicted infusion regimes to allow for accurate drug administration, the device may also be expected to have utility for other applications. For example, in investigations requiring the calculation of pharmacokinetic parameters, a TGI device may be useful for simplifying record keeping. When used in this fashion, a TGI device may not be required to calculate drug concentrations but rather to keep accurate logging information in terms of drug administration and times of administration, which may then be used to calculate the pharmacokinetic parameters.

For example, the TGI device may be useful for assisting in the determination of pharmacokinetic and pharmacodynamic parameters of pharmacological agents or drugs, and to provide statistical information for studies involving pharmacokinetic and pharmacodynamic parameters. It may also be useful for the construction, development, validation and comparison of pharmacokinetic and pharmacodynamic models based on such parameters.

Data capturing during clinical investigations may be facilitated by reducing the need to carry out other, typically more labour-intensive data capturing methods.

These additional fields of utility are discussed in further detail below:

### Compiling Statistical Information for Pharmacokinetic and Pharmacodynamic Studies

When new pharmacokinetic or pharmacodynamic models are developed, or during the development of new pharmacological agents or drugs, extensive research is required to evaluate drug disposition and movement in biological systems. This research may involve both animal and human studies. The accuracy of the results of such studies depends on keeping detailed drug administration logs and blood concentration measurements.

To assist in these studies, the TGI device may be configured to be operable in a tracking mode via a software-controlled utility. In such a configuration the TGI device may be operable to track drug administration and to provide detailed feedback information that can later be used for calculating pharmacokinetic parameters of the specific agent or drug being investigated.

The tracking may be performed by the measurement component of the TGI device. The tracking may, for example, be performed using the linear displacement sensor (potentiometer, etc.) and cooperating slider of the measurement component. The TGI device may be arranged such that the slider is engaged with the plunger flange of a syringe being used to perform the drug administration.

The feedback information provided by the TGI device can be relayed to a receiver via a wired or wireless connection in real-time. Instead or in addition it may captured by the TGI device for later downloading by a user. Once the researcher has obtained drug administration logs and enough blood samples to determine the temporal relationship of drug administration and resulting blood concentrations, a pharmacokinetic model can be constructed. This typically involves complex mathematical and statistical modelling performed by powerful computers.

In summary, the disclosed user-guidance device (TGI device) may find application in a method of compiling statistical information for pharmacokinetic and pharmacodynamic studies. The method may include monitoring infusion of a drug into a cohort of patients conducted by one or more manual drug infusion pump assemblies, each having a pump volume displacement parameter. The pump assemblies may, for example, be syringes driven by TCI pumps (or manually or by zero-order infusion pumps). For each patient within the cohort of patients, the method may include operating the measurement component of the disclosed TGI device to measure the pump volume displacement parameter of the pump assembly, and then operating the computing device of the TGI device to calculate a measured rate of infusion of the drug into the patient based on the measured pump volume displacement parameter. The method may further include recording data pertaining to the measured rate of infusion for each patient in the cohort.

The method may accordingly provide statistical information relating to measured rates of infusion pertinent to the patients in the cohort.

The method may further include recording times of measurement of the measured pump volume displacement parameter for each infusion, and/or the times at which the measured rates of infusion are calculated by the computing device. Thus, the method may include measuring and recording times of infusion of the drug into each patient within the cohort. The method may include logging these times as part of the statistical information that is provided by the method. Thus, the statistical information may additionally include times of infusion pertinent to the patients in the cohort.

The statistical information may be recorded, logged, disseminated and manipulated as part of a pharmacokinetic or pharmacodynamic study.

### Construction of Pharmacokinetic and Pharmacodynamic Models

As mentioned, the disclosed TGI device may also find application in the construction and development of pharmacokinetic and pharmacodynamic models. Accordingly, the invention extends to a method of operating the disclosed user-guidance device to construct pharmacokinetic and pharmacodynamic models. This method is summarised elsewhere in this specification.

### Comparison and Validation of Pharmacokinetic and Pharmacodynamic Models

As mentioned, the TGI device may also be operable to compare different pharmacokinetic and pharmacodynamic models with one another for purposes of validating them and assessing their accuracy. Thus, the TGI device may be configured to interrogate a pharmacokinetic model which is different than the model which the TCI pump is configured to interrogate, albeit that both models relate to the same drug. In such applications, the disclosed TGI device may be installed on or in association with a separate infusion device (such as an automated TCI pump) and be operable to track the infusion being carried out by that other device.

The TGI device may perform the tracking by using its linear displacement sensor and the cooperating slider of its measurement component. The TGI device may be arranged such that the slider is engaged with a flange of a syringe plunger driven or drivable by the TCI device.

During use of the TGI device in this arrangement, the slider may be caused to move linearly in sympathy with the syringe plunger, thereby tracking changing positions of the plunger flange along the longitudinal axis of the syringe barrel as the TCI pump performs its automated infusion.

The TGI device may accordingly be operable to track, record and display information on calculated blood- and/or brain concentrations as part of a comparative study of the two different models. If, for example, a new model has been developed for use in respect of a new target population (such as the elderly), investigations may be required to establish the variance of the real concentration of the drug in that population from the expected concentration.

In summary, and as disclosed elsewhere in this specification, the invention extends to a method of operating the disclosed user-guidance device for assessing the accuracy of first and second models selected from the group consisting of pharmacokinetic and pharmacodynamic models.

### Utility of TGI Device for Validation of Target Controlled Infusion (TCI) Pumps

The TGI device may also be useful for validating the accuracy of automated infusion devices such as Target Controlled Infusion (TCI) pumps. In such applications, the disclosed TGI device may be installed on or in association with a TCI pump and be operable to track the infusion of a drug by the TCI pump.

As in the former examples, the tracking may be performed by the measurement component of the TGI device. The TGI device may be arranged such that the slider is engaged with the flange of a syringe plunger driven or drivable by the TCI pump.

In such applications the TGI device may be configured to interrogate a similar pharmacokinetic model as that which the TCI pump is configured to interrogate, thereby to validate the operational accuracy of the TCI pump.

The TGI device may be operable to track, record and display information on calculated blood- or brain concentrations, and such information may be employed for comparison against corresponding concentration information calculated by the TCI device using the similar model.

Accordingly, the invention extends to a method, summarised elsewhere in this specification, of operating the disclosed user-guidance device for validation of the operational accuracy of an automated target-controlled infusion pump configured to drive a manual drug infusion pump assembly.

In all of the additional applications described above, the TGI device may be configured so that information which it derives can be relayed via a wired or wireless communication means to a receiver in near real time or be captured and stored by the TGI device for subsequent downloading by a user.

Throughout the specification and claims, and unless the context requires otherwise, the meaning of the term “engaged” or variations such as “engage” or “engages”—when used to refer to the engagement of the TGI's slider with the plunger flange of a syringe—shall not be restricted to direct engagement of the slider and flange but may also encompass indirect modes of engagement of the two components with each other. Indirect engagement may, for example, describe engagement of the slider and the flange with each other via an intermediary force transfer means. In the above examples, for example, when a TGI device is installed on or in association with a TCI pump, the slider of the TGI device may be configured to engage with a part of the TCI pump that is responsible for driving the syringe plunger, instead of being configured to engage directly with the flange of the syringe plunger. In such arrangements, a movement of the TCI pump's driving part may be correlated with a corresponding movement of the TGI device's slider. To this extent the slider and the syringe flange may be said to be engaged (indirectly) with each other.

The foregoing description has been presented for the purpose of illustration; it is not intended to be exhaustive or to limit the invention to the precise forms disclosed. Persons skilled in the relevant art can appreciate that many modifications and variations are possible in light of the above disclosure.

The description has been presented primarily in the context of anaesthesia, which is the preferred although not exclusive field of application of the system and method of the invention. It will be appreciated that other types of drugs may be suitable for infusion using the described system or TGI device or method. In certain circumstances the system, device and method may be applicable to the infusion of antibiotics in intensive care settings, or of antineoplastic agents in oncological settings.

Features and components of the described embodiments have been presented herein in certain configurations, arrangements and combinations that are pertinent to those particular embodiments. However, it will be apparent to those skilled in the art that such features and components may be implemented in other arrangements and combinations. In various embodiments, therefore, described features could instead be replaced by equivalent features.

Any of the steps, operations, components or processes described herein may be performed or implemented with one or more hardware or software units or program code portions, alone or in combination with other devices.

The described embodiments may be implemented with a computer program product comprising a non-transitory computer-readable medium containing computer program code, which can be executed by a processor for performing any or all of the steps, operations, or processes described. Software units or functions described in this application may be implemented as computer program code using any suitable computer language such as, for example, MicroPython™, Java™, C++, or Perl™ using, for example, conventional or object-oriented techniques. The exemplary embodiments of the TGI devices described herein employ MicroPython™ as the selected computer language, embodied as firmware.

The computer program code may be stored as a series of instructions, or commands on a non-transitory computer-readable medium, such as a random access memory (RAM), a read-only memory (ROM), a magnetic medium such as a hard-drive, or an optical medium such as a CD-ROM. Any such computer-readable medium may also reside on or within a single computational apparatus, and may be present on or within different computational apparatuses within a system or network.

Flowchart illustrations and block diagrams of methods, systems, and computer program products according to embodiments are used herein. Symbols or blocks of the flowchart illustrations and/or block diagrams, and combinations of symbols and blocks in the flowchart illustrations and/or block diagrams, may provide functions which may be implemented by computer readable program instructions. In some alternative implementations, the functions identified by the blocks may take place in a different order to that shown in the flowchart illustrations.

Some portions of this description describe the embodiments of the invention in terms of algorithms and symbolic representations of operations on information. These algorithmic descriptions and representations are commonly used by those skilled in the data processing arts to convey the substance of their work effectively to others skilled in the art. These operations, while described functionally, computationally, or logically, are understood to be implemented by computer programs or equivalent electrical circuits, microcode, or the like. The described operations may be embodied in software, firmware, hardware, or any combinations thereof.

The language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based hereon. Accordingly, the disclosure of the embodiments of the invention is intended to be illustrative, but not limiting, of the scope of the invention.

The systems, devices and methods described herein may provide certain advantages. Previously followed manually controlled infusion methods and regimens have been prone to errors in the calculation and implementation of required changes in infusion rate. The TGI devices disclosed herein may be able to solve the complex equations which describe the distribution of agents between compartments and allow for rapid adjustments in targets to achieve desired clinical effects. Advantageously, however, the clinician retains full control of the infusion process. The invention therefore provides many of the advantages of a TCI infusion device together with the benefits of the clinician retaining involvement based on experience.

The described TGI devices may be more flexible than TCI devices. Since the described devices do not automatically control the administration of anaesthesia and leave it to the clinician to administer the dose, the need for stringent regulatory approvals is obviated. TGI devices may therefore be less expensive to implement than TCI devices or systems which monitor actual blood concentration. Lower production costs may in turn make TGI devices suitable for healthcare systems which have limited financial resources. Less stringent regulation may also mean that TGI devices can benefit from the most recent PKPD models. These may account better for different patient populations (such as the elderly, the obese or the young) and have access to a greater library of patient profiles and drug interactions, so that the quality of information provided by TGI devices may be better than that provided by TCI devices. Furthermore, the need for clinicians to add drug dosage information to PK software in order to maintain accuracy can be obviated by the described TGI devices, since by their interaction with the syringe the devices take into account doses actually administered.

The described TGI devices may permit zero-order syringe pumps to be upgraded. Thus, a TGI device may serve as an after-market accessory which can be retro-fitted to an existing pump, thereby enhancing the functionality of the pump.

The described system can make allowance for variations in bolus dose quantities required to take account of patient-specific pain responses, or for special considerations associated with paediatric, elderly and obese patients. Administration of anaesthetic agents to those population groups may be expected to become safer and more predictable with the use of a TGI device.

Additional benefits may be provided in scenarios where suitable infusion devices are lacking but sedation or anaesthesia is required, for example on the battlefield, in emergency rooms or in remote locations (such as radiology or endoscopic suites) where access to fully equipped anaesthetic machines is costly.

Time-to-recovery or emergence may be more accurately predicted. Emergence of a patient within 12 minutes of a predicted emergence time has been shown to be possible in testing.

Throughout the specification and claims unless the context requires otherwise, the word “comprise” or variations such as “comprises” or “comprising” will be understood to imply the inclusion of a stated integer or group of integers but not the exclusion of any other integer or group of integers.

Also, the following terms will be understood to have the following meanings:
“BIS”—bispectral index;
“Bolus dose”—A quantity of drug given intravenously at a controlled, rapid rate;
“Ce”—effect-site concentration;
“Cp”—blood plasma concentration;
“Cpt”—targeted plasma concentration;
“Patient”—a patient selected from the group consisting of human and animal patients;
“PD”—pharmacodynamic;
“PK”—pharmacokinetic;
“PKPD”—either or both of pharmacokinetic and pharmacodynamic;
“TCI”—target-controlled infusion;
“TGI”—target-guided infusion;
“Zero-order infusion pump”—an infusion pump configured to be set to dispense a drug at a constant (zero-order) flow rate unless said rate is varied by manual adjustment. Pumps of this type are configured for manual adjustment of infusion flow rates but lack functionality to conduct infusion at automatically controlled rates. Without limitation thereto, a zero-order infusion pump may comprise a manually controllable, zero-order syringe pump.

### REFERENCES

1. Eleveld D J, Colin P, Absalom A R, Struys M M R F. Pharmacokinetic-pharmacodynamic model for propofol for broad application in anaesthesia and sedation. Br J Anaesth [Internet]. 2018 May; 120(5):942-59.
2. Scott J C, Cooke J E, Stanski D R. Electroencephalographic Quantitation of Opioid Effect: Comparative Pharmacodynamics of Fentanyl and Sufentanil. Anesthesiology. 1991; 74:34-42.
3. Gepts E, Shafer S L, Camu F, Stanski D R, Woestenborghs R, Van Peer A, et al. Linearity of Pharmacokinetics and Model Estimation of Sufentanil. Anesthesiology [Internet]. 1995 December; 83(6):1194-204.
4. Guay J, Gaudreault P, Goulet B. Pharmacokinetics of sufentanil in normal children. Can J Anesth. 1991; 39(1):14-20.
5. Herd, D, Anderson B J. Ketamine disposition in children presenting for procedural sedation and analgesia in a children's emergency department. Pediatr Anesth [Internet]. 2007 July; 17(7):622-9.
6. Delavenne X, Ollier E, Chollet S, Sandri F, Lanoiselée J, Hodin S, et al. Pharmacokinetic/pharmacodynamic model for unfractionated heparin dosing during cardiopulmonary bypass. Br J Anaesth [Internet]. 2017 May; 118(5):705-12.
7. Herd D W, Anderson B J, Keene N A, Holford N H. Investigating the pharmacodynamics of ketamine in children. Pediatr Anesth [Internet]. 2007 Nov. 12; 18(1).
8. Shibutani K, Inchiosa M A, Sawada K, Bairamian M. Pharmacokinetic mass of fentanyl for postoperative analgesia in lean and obese patients ††Presented in abstract form at the Annual Meeting of the American Society of Anesthesiologists, Las Vegas, Oct. 26, 2004. Br J Anaesth [Internet]. 2005 September; 95(3):377-83.
9. Hannivoort L N, Vereecke H E M, Proost J H, Heyse B E K, Eleveld D J, Bouillon T W, et al. Probability to tolerate laryngoscopy and noxious stimulation response index as general indicators of the anaesthetic potency of sevoflurane, propofol, and remifentanil. Br J Anaesth [Internet]. 2016 May; 116(5):624-31.
10. Slepchenko G, Simon N, Goubaux B, Levron J-C, Le Moing J-P, Raucoules-Aimé M. Performance of Target-controlled Sufentanil Infusion in Obese Patients. Anesthesiology [Internet]. 2003 January; 98(1):65-73.
11. Shafer S L, Gregg K M. Algorithms to rapidly achieve and maintain stable drug concentrations at the site of drug effect with a computer-controlled infusion pump. J Pharmacokinet Biopharm [Internet]. 1992 Apr. 1; 20(2):147-69.
12. Shafer S L, Varvel J R. Pharmacokinetics, Pharmacodynamics, and Rational Opioid Selection.

Anesthesiology [Internet]. 1991 January; 74(1):53-63.
13. Absalom A R, Lee M, Menon D K, Sharar S R, De Smet T, Halliday J, et al. Predictive performance of the Domino, Hijazi, and Clements models during low-dose target-controlled ketamine infusions in healthy volunteers. Br J Anaesth [Internet]. 2007 April; 98(5):615-23.
14. Minto C F, Schnider T W, Gregg K M, Henthorn T K, Shafer S L. Using the Time of Maximum Effect-site Concentration to Combine Pharmacokinetics and Pharmacodynamics. Anesthesiology [Internet]. 2003 August; 99(2):324-33.
15. Ginsberg B, Howell S, Glass P, Margolis J, Ross A, Dear G, et al. Pharmacokinetic Model-driven Infusion of Fentanyl in Children. Anesthesiology. 1996; 85:1268-75.
16. Shafer S L, Varvel J R, Aziz N, Scott J C. Pharmacokinetics of Fentanyl Administered by Computer-controlled Infusion Pump. Anesthesiology. 1990; 73:109-1102.
17. Minto C, Schnider T, Short T, Gregg K, Gentilini A, Shafer S. Response Surface Models for Anesthetic Drug Interactions. Anesthesiology. 2000; 92:1603-16.
18. Eleveld D J, Proost J H, Vereecke H, Absalom A R, Olofsen E, Vuyk J, et al. An Allometric Model of Remifentanil Pharmacokinetics and Pharmacodynamics. Anesthesiology [Internet]. 2017 June; 126(6):1005-18.
19. Short T G, Ho T Y, Minto C F, Schnider T W. Efficient trial design for eliciting a pharmacokinetic-pharmacodynamic model-based response surface. Anesthesiology. 2002; 96 (2): 400-8.
20. Shibutani K, Inchiosa M A, Sawada K, Bairamian M. Accuracy of pharmacokinetic models for predicting plasma fentanyl concentrations in lean and obese surgical patients: derivation of dosing weight (“pharmacokinetic mass”). Anesthesiology [Internet]. 2004; 101(3):603-13.
21. Pérus O, Marsot A, Ramain E, Dahman M, Paci A, Raucoules-Aimé M, et al. Performance of alfentanil target-controlled infusion in normal and morbidly obese female patients †. Br J

Anaesth [Internet]. 2012 October; 109(4):551-60.
22. Goresky G V, Koren G, Sabourin M A, Sale J P, Strunin L. The Pharmacokinetics of Alfentanil in Children. Anesthesiology. 1987; 67(5):654-9.

1-16. (canceled)

17. A user-guidance device for informing a required change to a pump volume displacement parameter of a manual drug infusion pump assembly, for use with manually-controlled infusion of at least one drug into a patient, said user-guidance device comprising:
at least one measurement component arranged to measure the pump volume displacement parameter; and
a computing device arranged to:
calculate a measured rate of infusion of the drug into the patient based on the measured pump volume displacement parameter;
calculate with reference to said measured rate of infusion and to at least one model selected from the group consisting of pharmacokinetic and pharmacodynamic models, a model-simulated drug concentration in said patient;
derive user-guidance information based on said model-simulated drug concentration; and
output the user-guidance information.

18. A drug infusion system which includes a user-guidance device as claimed in claim 17 and a manual drug infusion pump assembly.

19. The drug infusion system as claimed in claim 18 wherein the manual drug infusion pump assembly is selected from the group consisting of syringes and non-pharmacokinetic, zero-order syringe pumps in combination with syringes.

20. The drug infusion system as claimed in claim 18 wherein the computing device is further arranged to calculate, with reference to the pump volume displacement parameter and the model, a rate of infusion required for achieving in said patient a drug concentration within a target concentration range.

21. The drug infusion system as claimed in claim 20 wherein the computing device is arranged to compare the measured rate of infusion against the required infusion rate, thereby to establish a deviation of the measured infusion rate from the required infusion rate; and generate deviation notification data if said deviation breaches a definable threshold.

22. The drug infusion system as claimed in claim 18 wherein the computing device is arranged to generate deviation notification data if the model-simulated drug concentration breaches a definable threshold concentration.

23. The drug infusion system as claimed in claim 21, which further includes at least one alerting component configured to trigger a user-discernible alert signal responsive to the generation of the deviation notification data.

24. The drug infusion system as claimed in claim 18 wherein the measurement component and the computing device are of a unitary construction configured for tool-free engagement with, and disengagement from, the pump assembly.

25. The drug infusion system as claimed in claim 18 wherein the drug is selected from the group consisting of anaesthetic, hypnotic, analgesic, and neuromuscular blocking agents.

26. The drug infusion system as claimed in claim 18, which comprises a plurality of pump assemblies for concurrently infusing a plurality of drugs, and a plurality of measurement components arranged in data communication with the computing device, and wherein the computing device is arranged to calculate
data relating to interactions between the drugs, and
with reference to said data, a required change to a rate of infusion of at least one of the drugs.

27. The drug infusion system as claimed in claim 18 wherein the computing device includes a processing component and a memory configured to provide computer program instructions as software units to the processor to execute the calculations.

28. A computer implemented method for informing a required change to a pump volume displacement parameter of a manual drug infusion pump assembly, the method including:
monitoring at least one manual drug infusion pump assembly having a pump volume displacement parameter;
measuring said pump volume displacement parameter;
providing a computing device and operating it to:
calculate a measured rate of infusion of a drug into a patient based on said pump volume displacement parameter;
calculate with reference to said measured rate of infusion and to at least one model selected from pharmacokinetic and pharmacodynamic models, a model-simulated drug concentration in said patient;
derive user-guidance information based on said model-simulated drug concentration; and
output the user-guidance information for control of manual drug infusion.

29. The method as claimed in claim 26, which includes generating deviation notification data if either or both of the following conditions are met:
(a) the model-simulated drug concentration breaches a definable threshold concentration;
(b) a deviation of the measured rate of infusion from a required infusion rate occurs, said required infusion rate being a rate of infusion required for achieving, in said patient, a drug concentration within a target concentration range, and said deviation of the measured rate of infusion therefrom being established by performance of the following steps:
calculating, with reference to the pump volume displacement parameter and the model, the required infusion rate; and
comparing the measured rate of infusion against the required infusion rate, thereby to establish the deviation of the measured rate of infusion from the required infusion rate.

30. The method as claimed in claim 29, which further includes generating deviation notification data if the model-simulated drug concentration breaches a definable threshold concentration.

31. The method as claimed in claim 29, which includes
monitoring infusion of a plurality of drugs from a plurality of pump assemblies;
measuring respective volumes of the drugs dispensed from the respective pump assemblies;
operating the computing device to calculate information relating to interactions between said drugs, based on the measured volumes dispensed; and
outputting said information.

32. A method of operating a user-guidance device as claimed in claim 17 to compile statistical information for a study selected from the group consisting of pharmacokinetic and pharmacodynamic studies; said method including steps of
monitoring infusion of a drug into a cohort of patients conducted by a manual drug infusion pump assembly, said pump assembly having a pump volume displacement parameter;
for each patient within said cohort of patients:
operating the measurement component of the user-guidance device to measure the pump volume displacement parameter of the pump assembly;
operating the computing device of the user-guidance device to calculate a measured rate of infusion of the drug into the patient based on the measured pump volume displacement parameter, and
recording data pertaining to the measured rate of infusion;
thereby to provide statistical information relating to measured rates of infusion pertinent to the patients in the cohort.

33. A method of operating a user-guidance device as claimed in claim 17 to construct a model selected from the group consisting of pharmacokinetic and pharmacodynamic models; said method including steps of
monitoring infusion of a drug into a cohort of patients conducted by a manual drug infusion pump assembly, said pump assembly having a pump volume displacement parameter;
for each patient within said cohort of patients:
operating the measurement component of the user-guidance device to measure the pump volume displacement parameter of the pump assembly;
operating the computing device of the user-guidance device to calculate a measured rate of infusion of the drug into the patient based on the measured pump volume displacement parameter;
recording data pertaining to the measured rate of infusion;
measuring and recording a time of infusion of the drug into the patient; and
measuring and recording a concentration of the drug in the patient;
thereby to provide statistical information relating to measured rates of infusion, times of infusion and drug concentrations pertinent to the patients in the cohort; and
mathematically manipulating the statistical information using a pre-defined algorithm adapted to cross-reference parameters selected from the group consisting of the measured rates of infusion, the times of infusion and the drug concentrations for the patients in the cohort, thereby to construct the model.

34. A method of operating a user-guidance device as claimed in claim 17 for assessing accuracy of first and second models selected from the group consisting of pharmacokinetic and pharmacodynamic models; said method including steps of
monitoring infusion of a drug into a patient conducted by a manual drug infusion pump assembly operable to achieve a target drug concentration in the patient calculated by interrogation of the first model, said pump assembly having a pump volume displacement parameter;
operating the measurement component of the user-guidance device to measure the pump volume displacement parameter of the pump assembly;
operating the computing device of the user-guidance device to
calculate a measured rate of infusion of the drug into the patient based on the measured pump volume displacement parameter; and
calculate with reference to said measured rate of infusion and to the second model, a model-simulated drug concentration in said patient;
measuring and recording a concentration of the drug in the patient;
comparing the measured concentration of the drug in said patient with the target drug concentration and the model-simulated drug concentration; thereby to provide comparative information relating to the first and second models; and
outputting the comparative information to support assessment of the relative accuracy of the first and second models in relation to the measured concentration.

35. A method of operating a user-guidance device as claimed in claim 17 for validation of operational accuracy of an automated target-controlled infusion pump configured to drive a manual drug infusion pump assembly for infusing a drug into a patient to achieve a target drug concentration; said method including steps of
monitoring the infusion of the drug into the patient by operating the measurement component of the user-guidance device to measure a pump volume displacement parameter of the pump assembly;
operating the computing device of the user-guidance device to
calculate a measured rate of infusion of the drug into the patient based on the measured pump volume displacement parameter;
calculate with reference to said measured rate of infusion and to at least one model selected from the group consisting of pharmacokinetic and pharmacodynamic models, a model-simulated drug concentration in said patient;
comparing the target drug concentration against the model-simulated drug concentration calculated by the user-guidance device, thereby to determine a deviation of the target concentration from the model-simulated concentration and provide a metric of the operational accuracy of said target-controlled infusion pump; and
outputting said metric to support the validation of the operational accuracy of said target-controlled infusion pump.
